SEQUENCE LISTING (26 sequences)

SEQ ID NO: 1 - protein, 9 aa, Drosophila melanogaster
KRKESYAIY

SEQ ID NO: 2 - protein, 9 aa, Homo sapiens
SRKESYSIY

SEQ ID NO: 3 - protein, 13 aa, Drosophila melanogaster
MFLEDLKQIVDNS

SEQ ID NO: 4 - protein, 39 aa, Drosophila melanogaster
PPKTSGKAAKKAGKAQKNITKTDKKKKRKAKESYAIYIY

SEQ ID NO: 5 - protein, 12 aa, Artificial sequence
  Consensus sequence
XXXXDXKXXXXX

SEQ ID NO: 6 - protein, 13 aa, Drosophila melanogaster
FIHRDIKPDNFLM

SEQ ID NO: 7 - protein, 13 aa, Drosophila melanogaster
LIHRDLKPSNIFF

SEQ ID NO: 8 - protein, 13 aa, Drosophila melanogaster
IIHRDLKPSNIVV

SEQ ID NO: 9 - protein, 44 aa, Drosophila melanogaster
PPKTSGKAAKKAGKAQKNITKTDKKKKRKRKESYAIYIYKVLKQ

SEQ ID NO: 10 - protein, 47 aa, Homo sapiens
PEPSKSAPAPKKGSKKAITKAQKKDGKKRKRSRKESYSIYVYKVLKQ

SEQ ID NO: 11 - protein, 64 aa, Homo sapiens
PRSPSKSSPRKGSPRKGSPRKGSPKRGGKGAKRAGKGGRRRNVVKRRRRRRESYGSYIYRVLKQ

SEQ ID NO: 12 - protein, 47 aa, Rattus sp.
PEPAKSRPAPKKGSKKAVTKAQKKDGKERKRSRKESYSVYVYKVLKQ

SEQ ID NO: 13 - protein, 47 aa, Xenopus laevis
PEPAKSAPAPKKGSKKAVTKTPKKDGKKRRKSRKESYAIYVYKVMKQ

SEQ ID NO: 14 - protein, 44 aa, Caenorhabditis elegans
PPKPSAKGAKKAAKTVVAKPKDGKKRRHARKESYSVYIYRVLKQ

SEQ ID NO: 15 - protein, 35 aa, Euplotes crassus
PATPAKRAKRVQQEKRHHKKRTETFSVYIYRVLKQ

SEQ ID NO: 16 - protein, 50 aa, Saccharomyces cerevisiae
SSAAEKKPASKAPAEKKPAAKKTSTSVDGKKRSKVRKETYSSYIYKVLKQ

SEQ ID NO: 17 - protein, 46 aa, Schizosaccharomyces pombe
SAAEKKPASKAPAGKAPRDTMKSADKKRGKNRKETYSSYIYRVLKQ

SEQ ID NO: 18 - protein, 68 aa, Arabidopsis thaliana
EKKPAEKKPAAEKPVEEKSKAEKAPAEKKPKAGKKLPKEAGAGGDKKKKMKKKSVETYKIYIFKVLKQ

SEQ ID NO: 19 - protein, 34 aa, Trypanosoma brucei
ATPKSTPAKTRKEAKKTRRHQATWNVYVSRSLRS

SEQ ID NO: 20 - protein, 638 aa, Artificial sequence
  Consensus sequence
LTPVSAKVVKDYYKVVTKPMDLQTLRENVRQRLYTSREEFLEDLELIVDNSAIYNGPQSALTLAAQSMLSLCDELLAEKEDKLARLEKAINPLLDDDDQVALSFILDNLVSQKIKAVPDSWPFLKPVNKKQVKDYYTVIVNPMDLETIGKNISAHKYQSRASFLADVELIAANSVQYNGSDSQYTKTAQEIVEVAQTTLIEFSEHLGQLENDIATAQEAALEEAELESLDEAWGNDDYNFTRGSRASSPGDLYIDVEGHGGDASSSLSISRSAGAEAGSSNTAPAVRKPAPPGPGEVKRGRGRPRKQRDPVLDVKSATPVKRGRGRPRKDSLASNMSHTQAYFLDEDLQCSTDDEDDDEEEDFQEVSEDGEDGAGILADGEEGTAQADAASGLFDPKNIKTDLLLSAGEDAEEDAGSDDSGDVAEAAVQLSGVGGYYAQDSDVGSGGVDPNYDPSDFLAMHKQRQSLGENSSLDMGAETSFLSHEGDGGGASNGAEASTIAAGGADLGMDASMAMQMAPDMKVNTQDTGFGIIGGLDVSESDEDDDGSRVRIKKEVFDDGDYALEEEQRGGAAVLSQVHLVDSSEDSTTLDYQQPPQLDFQSVAGDSDLDSDVMPPMQSEQLQQQQTPQGDNDYAWTF

SEQ ID NO: 21 - protein, 634 aa, Drosophila melanogaster
LFPVSAKKVPDYYRVVTKPMDLQTMREYIRQRRYTSREMFLEDLKQIVDNSLIYNGPQSAYTLAAQRMFSSCFELLAEREDKLMRLEKAINPLLDDDDQVALSFIFDKLHSQIKQLPESWPFLKPVNKKQVKDYYTVIKRPMDLETIGKNIEAHRYHSRAEYLADIELIATNCEQYNGSDTRYTKFSKKILEYAQTQLIEFSEHCGQLENNIAKTQERARENAPEFDEAWGNDDYNFDRGSRASSPGDDYIDVEGHGGHASSSNSIHRSMGAEAGSSHTAPAVRKPAPPGPGEVKRGRGRPRKQRDPVEEVKSQNPVKRGRGRPRKDSLASNMSHTQAYFLDEDLQCSTDDEDDDEEEDFQEVSEDENNAASILDQGERINAPADAMDGMFDPKNIKTEIDLEAHQMAEEPIGEDDSQQVAEAMVQLSGVGGYYAQQQQDESMDVDPNYDPSDFLAMHKQRQSLGEPSSLQGAFTNFLSHEQDDNGPYNPAEASTSAASGADLGMDASMAMQMAPEMPVNTMNNGMGIDDDLDISESDEEDDGSRVRIKKEVFDDGDYALQHQQMGQAASQSQIYMVDSSNEPTTLDYQQPPQLDFQQVQEMEQLQHQVMPPMQSEQLQQQQTPQGDNDYAWTF

SEQ ID NO: 22 - protein, 467 aa, Homo sapiens
HTPVNAKVVKDYYKIITRPMDLQTLRENVRKRLYPSREEFREHLELIVKNSATYNGPKHSLTQISQSMLDLCDEKLKEKEDKLARLEKAINPLLDDDDQVAFSFILDNIVTQKMMAVPDSWPFHHPVNKKFVPDYYKVIVNPMDLETIRKNISKHKYQSRESFLDDVNLILANSVKYNGPESQYTKTAQEIVNVCYQTLTEYDEHLTQLEKDICTAKEAALEEAELESLDPMTPGPYTPQPPDLYDTNTSLSMSRDASVFQDESNMSVLDIPSATPEKQVTQEGEDGDGDLADEEEGTVQQPQASVLYEDLLMSEGEDDEEDAGSDEEGDNPFSAIQLSESGSDSDVGSGGIRPKQPRMLQENTRMDMENEESMMSYEGDGGEASHGLEDSNISYGSYEEPDPKSNTQDTSFSSIGGYEVSEEEEDEEEEEQRSGPSVLSQVHLSEDEEDSEDFHSIAGDSDLDSDE

SEQ ID NO: 23 - protein, 135 aa, Drosophila melanogaster
ARTKQTARKSTGGKAPRKQLATKAARKSAPATGGVKKPHRYRPGTVALREIRRYQKSTELLIRKLPFQRLVREIAQDFKTDLRFQSSAVMALQEASEAYLVGLFEDTNLCAIHAKRVTIMPKDIQLARRIRGERA

SEQ ID NO: 24 - protein, 134 aa, Homo sapiens
ARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGEA

SEQ ID NO: 25 - DNA, 35 bp, Artificial sequence
  synthetic oligonucleotide primer
gagatgttcctcgaggctctcaagcagattgtggt

SEQ ID NO: 26 - protein, 9 aa, Drosophila melanogaster
ESYAIYIYK

(19) United States
(12) Patent Application Publication
(10) Pub. No.: US 2006/0115482 A1
(43) Pub. Date: Jun. 1, 2006

(54) Modifications of histone proteins as indicators of cell proliferation and differentiation

(75) Inventors: Frank Sauer, Riverside, CA (US); David A. Wassarman, Middleton, WI (US); Tobias Maile, Riverside, CA (US)

Correspondence Address: QUINE INTELLECTUAL PROPERTY LAW GROUP, P.C., P O BOX 458, ALAMEDA, CA, 94501, US

(73) Assignees: The Regents of the University of California; Wisconsin Alumni Research Foundation

(21) Appl. No.: 11/116,142

(22) Filed: Apr. 26, 2005

Related U.S. Application Data
(60) Provisional application No. 60/565,968, filed on Apr. 27, 2004.
(60) Provisional application No. 60/568,299, filed on May 4, 2004.
(60) Provisional application No. 60/571,311, filed on May 13, 2004.

(51) Int. Cl. G01N 33/574; C07H 21/04; A61K 39/395; C12N 5/06

(52) U.S. Cl. 424/155.1; 435/7.23; 435/338; 435/320.1; 530/388.8; 536/23.53

(57) ABSTRACT

The invention provides a method of a method of characterizing the proliferative state of cells in a biological sample based on detecting transcription factor-mediated phosphorylation of histone H2B (H2B) at residue 36 (numbered according to human H2B). This method optionally includes detection of transcription factor-mediated acetylation of histone H3 (H3) at residue 14 and/or acetylation of histone H4, which also provides an indication of cell proliferation. The invention also provides antibodies specific for H2B phosphorylated at H2B; transcription factor and H2B derivatives and related polynucleotides, vectors, host cells, recombinant production methods and compositions; and screening methods for modulators of H2B phosphorylation at residue 36.

### CROSS-REFERENCE TO RELATED APPLICATIONS

This application claims the benefit of the following U.S. provisional applications: Ser. No. 60/565,968 filed on Apr. 27, 2004, Ser. No. 60/568,299 filed May 4, 2004 and Ser. No. 60/571,311 filed May 13, 2004. Each of the applications cited above is incorporated by reference in its entirety.

### STATEMENT AS TO RIGHTS TO INVENTIONS MADE UNDER FEDERALLY SPONSORED RESEARCH AND DEVELOPMENT

This invention was made with government support under grant no. 1 R01 GM66204-01. The Government may have certain rights in the invention.

### FIELD OF THE INVENTION

This invention relates generally to the detection of modifications of histone proteins as indicators of cell proliferation and differentiation. More particularly, the invention relates to phosphorylation and acetylation of histones and related methods and compositions.

### BACKGROUND OF THE INVENTION

Transcription initiation in eukaryotes involves dynamic changes in chromatin structure that permit assembly of the transcription machinery at a gene promoter (1, 2). The fundamental structural unit of chromatin is the nucleosome, which contains 146 base pairs of DNA wrapped around a histone octamer comprised of two copies each of histones H2A, H2B, H3, and H4 (3). Distinct patterns of histone modifications (e.g. acetylation, phosphorylation, and methylation) may act as ‘modification cassettes’ that facilitate DNA-dependent events (4, 5). For example, in vertebrates phosphorylation of H2B serine 14 is associated with apoptotic chromatin, and in all eukaryotes phosphorylation of H3 serine 10 is associated with transcriptionally active and mitotic chromatin (4-6). Although all histones are phosphorylated in vivo, the function of many of these modifications and the kinases that carry them out were not known (7).

### SUMMARY OF THE INVENTION

The invention provides a method of method of characterizing the proliferative state of cells in a biological sample. The sample can be from any organism, but is preferably from a mammal, and more preferably from a human. The method entails detection of phosphorylation of histone H2B (H2B) at residue 36, wherein H2B residues are numbered according to human H2B, and wherein phosphorylation of H2B residue 36 is an indicator of cell proliferation. H2B residue 36 can be a serine or a threonine. The detection of phosphorylation of H2B at residue 36 preferably includes: (a) contacting a biological sample comprising H2B with an antibody specific for H2B having a phosphate on H2B residue 36 under conditions suitable for antibody binding; and (b) detecting H2B antibody binding.

In preferred embodiments, the method additionally includes determining whether H2B that is phosphorylated at H2B residue 36 (H2B-r36p) is physically associated with the promoter of a gene. This determination can conveniently be made using in vivo cross-linked chromatin immunoprecipitation.

According to this method, H2B antibody binding is detected as an indication of the level of transcriptional activation of one or more genes, such as for example, cell cycle genes (e.g., those that encode proteins that contribute to progression through the G2/M phase of the cell cycle, like human cdc25), genes that participate in tissue differentiation, and oncogenes.

In particular embodiments, the amount of H2B-r36p physically associated with the promoter in a test sample is compared with the amount of H2B-r36p physically associated with the promoter in a control sample. Tissue biopsies, especially of a tissue suspected of being cancerous, can, for example, be analyzed in this manner. In this case, the difference between the amount of H2B-r36p physically associated with the promoter in the test sample and the amount of H2B-r36p physically associated with the promoter in the control sample provides a metric useful in the diagnosis and/or prognosis of cancer.

In a variation of the method, the biological sample comprises histone H3 (H3) and/or histone H4 (H4), and the method additionally comprising detection of acetylation of histone H3 at residue 14, wherein H3 residues are numbered according to human H3, and/or acetylation of histone H4. This variation of the method can be employed when the sample comprises H3 and H4, in addition to H2B. Acetylation of H3 residue 14 and acetylation of H4 are indicators of cell proliferation. The detection of either or both of these acetylation modifications preferably includes: (a) contacting a biological sample with an antibody specific for H3 having an acetyl group on H3 residue 14 and/or with an antibody specific for acetylated H4 under conditions suitable for antibody binding; and (b) detecting H3 and/or H4 antibody binding.

In preferred embodiments, the method additionally includes determining whether H3 that is acetylated at H3 residue 14 (H3-r14a) and/or acetylated H4 is/are physically associated with the promoter of a gene. This determination can conveniently be made using in vivo cross-linked chromatin immunoprecipitation.

According to this variation of the method, H3 and/or H4 antibody binding is detected as an indication of the level of transcriptional activation of one or more genes, such as for example, cell cycle genes (e.g., those that encode proteins that contribute to progression through the G1 or G2/M phase of the cell cycle, like human cdc25), genes that participate in tissue differentiation, and oncogenes.

In particular embodiments, the amount of H3-r14a and/or acetylated H4 physically associated with the promoter in a test sample is compared with the amount of H2B-r36p physically associated with the promoter in a control sample. Tissue biopsies, especially of a tissue suspected of being cancerous, can, for example, be analyzed in this manner. In this case, the difference between the amount of H3-r14a and/or acetylated H4 physically associated with the promoter in the test sample and the amount of H3-r14a and/or acetylated H4 physically associated with the promoter in the control sample provides a metric useful in the diagnosis and/or prognosis of cancer.

Another method of the invention facilitates characterization of the transcriptional activity of a gene in a biological sample. This method entails determining whether histone H2B (H2B) that is phosphorylated at H2B residue 36 (H2B-r36p) is physically associated with the promoter of the gene, wherein physical association of H2B-r36p with the promoter is an indicator of transcriptional activation of the gene. H2B residues are numbered according to human H2B. In a variation of this method, the biological sample comprises histone H3 (H3) and/or histone H4 (H4), and the method additionally entails determining whether H3 that is acetylated at H3 residue 14 (H3-r14a) and/or acetylated H4 is/are physically associated with the promoter of a gene. H3 residues are numbered according to human H3. The physical association of H3-r14a and/or acetylated H4 with the promoter is/are indicators of transcriptional activation.

Additionally, the invention provides a method for identifying one or more eukaryotic promoters in a biological sample. The method entails isolating from the sample one or more polynucleotides that are physically associated in vivo with histone H2B (H2B) that is phosphorylated at H2B residue 36 (H2B-r36p). H2B residues are numbered according to human H2B. In preferred embodiments, the isolation of one or more polynucleotides that are physically associated in vivo with H2B-r36p is carried out by in vivo cross-linked chromatin immunoprecipitation. Using this technique, a plurality of different polynucleotides is typically isolated. Any isolated polynucleotides can optionally be further analyzed, preferably by hybridization to one or more known polynucleotides. This analysis can be conducted, for example, by hybridization of the isolated polynucleotides to one or more known polynucleotides that are arrayed in a DNA microarray.

The invention also provides an antibody specific for histone H2B (H2B) comprising a phosphate on H2B residue 36, wherein H2B residues are numbered according to human H2B. The antibody can be specific for H2B from any organism, but specificity for a mammalian H2B is preferred, with specificity for human H2B being particularly preferred. H2B residue 36 can be a serine or a threonine. The antibody can be polyclonal, monoclonal, or an antibody fragment or derivative, such as a Fab, a (Fab′)₂, a single chain Fv (scFv), and a (scFv′)₂. The antibody can be from any immunoglobulin class, but is preferably an IgG or is derived therefrom.

Another aspect of the invention is a method of producing an antibody of the invention. The method entails: (a) administering a histone H2B (H2B), or a fragment thereof, to a mammal, wherein the H2B or H2B fragment comprises phosphorylated H2B residue 36, the H2B residues being numbered according to human H2B, and wherein said administration elicits an immune response; and (b) recovering an antiserum or spleen cells from the mammal. In a preferred embodiment, spleen cells are recovered from the animal and used to produce one or more hybridomas.

The invention also provides a kit including an article containing the anti-H2B-r36p antibody of the invention. The kit can include instructions for carrying out the above-described method of characterizing the proliferative state of cells in a biological sample. In preferred embodiments, the kit additionally includes another article containing histone H2B (H2B), or a fragment thereof, wherein the H2B or H2B fragment comprises phosphorylated H2B residue 36, the H2B residues being numbered according to human H2B.

In addition, or in the alternative, the kit can include an article containing an antibody specific for acetylated histone H3 (H3) and/or acetylated histone H4 (H4). The anti-acetylated H3 antibody is preferably specific for H3 comprising an acetyl group on H3 residue 14, wherein H3 residues are numbered according to human H3. In preferred variations of this embodiment, the kit includes another article containing a polypeptide to which the anti-acetylated H3 or H4 antibody will bind. Thus, kits containing an anti-H3-r14a antibody also preferably contain a histone H3 (H3), or a fragment thereof, wherein the H3 or H3 fragment comprises acetylated H3 residue 14, the H3 residues being numbered according to human H3. Similarly, kits containing an anti-acetylated H4 antibody preferably contain an acetylated histone H4 or a fragment thereof.

Another aspect of the invention is an isolated polypeptide including a fragment of a transcription factor, wherein the fragment includes a double bromodomain kinase and phosphorylates histone H2B (H2B) at H2B residue 36, wherein H2B residues are numbered according to human H2B. The polypeptide generally does not include more than about 650 contiguous amino acids of the transcription factor. The transcription factor fragment can phosphorylate a serine, a threonine, or either at residue 36. In preferred embodiments, the fragment includes a serine/threonine kinase domain and a double ATP binding motif. The transcription factor can be from any organism, but is preferably from a mammal, and more preferably from a human, e.g., human TAF1.

The invention also provides an isolated polypeptide including a fragment of a histone H2B (H2B), wherein the fragment comprises H2B residue 36, the H2B residues being numbered according to human H2B. The polypeptide generally does not comprise more than about 40 contiguous amino acids of the H2B. H2B residue 36 can be a serine or a threonine. The H2B can be from any organism, but is preferably from a mammal, and more preferably from a human. In one embodiment, the polypeptide does not include more than about 10 contiguous amino acids of the H2B. For example, the H2B fragment can comprise the Drosophila amino acid sequence KRKESYAIY (SEQ ID NO:______) or the human amino acid sequence SRKESYSIY (SEQ ID NO:______). The H2B fragment can be phosphorylated or unphosphorylated at residue 36.

Other aspects of the invention include polynucleotides, vectors, host cells, and related recombinant production methods. Thus, the invention provides an isolated polynucleotide that encodes a transcription factor polypeptide of the invention, wherein the polynucleotide does not contain more than about 1950 contiguous nucleotides of transcription factor coding sequence. The invention also provides an isolated polynucleotide that encodes an H2B polypeptide of the invention, wherein the polynucleotide does not comprise more than about 120 contiguous nucleotides of histone H2B coding sequence. Also provided are a vector that includes either or both of these polynucleotides, which, in preferred embodiments, is an expression vector, and a host cell including the vector. Either or both of the encoded polypeptides can be produced by (a) culturing the host cell under conditions suitable for expression of the polypeptide; and (b) recovering the expressed polypeptide from the culture.

In addition, the invention provides a method of prescreening for a modulator of phosphorylation of histone H2B (H2B) at residue 36 or a modulator of promoter association of H2B that is phosphorylated at residue 36 (H2B-r36p), based on assaying binding of test agents to an H2B polypeptide or polynucleotide or a transcription factor polypeptide or polynucleotide. The method entails: (a) contacting a test agent with a polypeptide selected from the group consisting of an H2B, or a fragment thereof, or a transcription factor, or a fragment thereof, or with a polynucleotide encoding the polypeptide; and (b) detecting specific binding of the test agent to the polypeptide or polynucleotide.

The H2B fragment includes H2B residue 36, which can be a serine or a threonine, the H2B residues being numbered according to human H2B. The H2B can be from any organism, but is preferably from a mammal, and more preferably from a human.

The transcription factor fragment includes a double bromodomain kinase and phosphorylates H2B at H2B residue 36. The transcription factor can be from any organism, but is preferably from a mammal, and more preferably from a human.

The invention also provides a method of screening for a modulator of phosphorylation of histone H2B (H2B) at residue 36. The method entails: (a) contacting a test agent with a medium including H2B, or a fragment thereof, and a transcription factor, or a fragment thereof, under conditions suitable for phosphorylation of H2B residue 36; and (b) detecting phosphorylation of H2B residue 36. The detection of phosphorylation of H2B at residue 36 preferably includes: (a) contacting the H2B, or fragment thereof, with an antibody specific for H2B comprising a phosphate on H2B residue 36 under conditions suitable for antibody binding; and (b) detecting H2B antibody binding. Generally, any phosphorylation of H2B residue 36 is compared with phosphorylation of H2B residue 36 in the absence of test agent or in the presence of a lower amount of test agent than in (a).

In an alternative embodiment, the invention provides a method of screening for a modulator of promoter association of histone H2B (H2B) that is phosphorylated at residue 36 (H2B-r36p). The method entails: (a) contacting a test agent with cells comprising H2B, or a fragment thereof, and a transcription factor, or a fragment thereof; and (b) determining whether H2B-r36p is physically associated with the promoter of a gene. This determination preferably includes in vivo cross-linked chromatin immunoprecipitation. In preferred embodiments, the gene is a cell cycle gene, a tissue differentiation gene, or an oncogene. Generally, any promoter association of H2B-r36p is compared with the promoter association of H2B-r36p in the absence of test agent or in the presence of a lower amount of test agent than in (a).

In the screening methods of the invention, the H2B fragment includes H2B residue 36, which can be a serine or a threonine, the H2B residues being numbered according to human H2B. The H2B can be from any organism, but is preferably from a mammal, and more preferably from a human.

The transcription factor fragment includes a double bromodomain kinase and phosphorylates histone H2B (H2B) at H2B residue 36. The transcription factor can be from any organism, but is preferably from a mammal, and more preferably from a human.

The prescreening and screening methods of the invention can additionally include the recordation of: (a) any modulator of phosphorylation of H2B at residue 36, or any modulator of promoter association of H2B-r36p, in a database of candidate agents that may modulate cell proliferation; (b) any inhibitor of phosphorylation of H2B at residue 36, or any inhibitor of promoter association of H2B-r36p, in a database of candidate agents that may inhibit cell proliferation; and/or (c) the recordation of any test agent that stimulates phosphorylation of H2B at residue 36, or that stimulates of promoter association of H2B-r36p, in a database of candidate agents that may stimulate cell proliferation.

The prescreening and screening methods optionally include determining whether the test agent inhibits or stimulates cell proliferation in an in vitro or in vivo assay.

The invention additionally provides a method of modulating cell proliferation that entails contacting cells comprising a transcription factor and histone H2B (H2B) with an effective amount of a phosphorylation modulator. The transcription factor is one that includes a double bromodomain kinase and phosphorylates H2B at H2B residue 36, wherein H2B residues are numbered according to human H2B. The modulator is an agent that reduces or increases phosphorylation. An effective amount of the modulator is an amount sufficient to inhibit cell proliferation, in the case of an agent that reduces phosphorylation, or to stimulate cell proliferation, in the case of an agent that increases phosphorylation. The cells can be from any organism that expresses the transcription factor and histone H2B, although mammalian cells are preferred, with human cells being more preferred. The cells can be in vitro or in vivo. In an exemplary in vivo embodiment, the modulator is an inhibitor that reduces H2B-r36 phosphorylation, and the inhibitor is contacted with the cells by administering a composition including the inhibitor to a cancer patient. In an alternative in vivo embodiment, the modulator is a stimulatory agent that increases H2B-r36 phosphorylation, and the stimulatory agent is contacted with the cells by administering a composition including the agent to a subject having a condition treatable by induction of cell proliferation.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1. The TAF1 CTK phosphorylates histone H2B. (A) Silver stained gel of immunopurified TAF1 and endogenous Drosophila melanogaster TFIID. The asterisk indicates the position of the antibody heavy chain. (B) Coomassie blue stained gel (left) and corresponding autoradiogram (right) of in vitro kinase assays programmed with γ³²P-ATP and recombinant histones (H1, H2A, H2B, H3, and H4) in the presence (+) or absence (−) of immunopurified TAF1 or TFIID. (C) Coomassie blue stained gel (top) and corresponding autoradiogram (bottom) of in vitro kinase assays as in (B) but containing the indicated recombinant histones, and denatured/renatured TAF1. (D) Schematic representation of TAF1 and the CTK. Positions of the NH₂-terminal kinase domain (NTK), the histone acetyltransferase domain (HAT), the ubiquitin activating/conjugating domain (E1/E2), the double bromodomain (B1 and B2), and the CTK are indicated. (E) Coomassie blue stained gel (top) and corresponding autoradiogram (bottom) of in vitro kinase assays as in (B) except that reactions were programmed with recombinant CTK or NTK and either H2B or RAP74.

FIG. 2. The TAF1 CTK phosphorylates serine 33 in H2B. (A) Schematic representation of TAF1 and TAF1 derivatives. The positions of enzymatic domains are indicated (see FIG. 1D). The position and amino acid sequence of the S/T-kinase motif and the positions of the ATP-binding motifs and CTK(D1538A) mutation are indicated. (B) Autoradiograms of in vitro kinase assays containing γ³²P-ATP, native nucleosomes (top) or recombinant H2B (bottom), and TFIID, TAF1, or TAF1 derivatives. (C) Coomassie blue stained gel (top) and corresponding autoradiogram (bottom) of in vitro kinase assays programmed with γ³²P-ATP, CTK, and H2B, tailless H2B core-domain (H2B-core), or H2BT peptide (H2BT). (D) Schematic representation of H2BT (top). Serines (S) are highlighted in yellow. Autoradiograms (A) and corresponding coomassie blue stained gels (C) of in vitro kinase assays containing TAF1, γ³²P-ATP, and increasing amounts of H2BT or H2BT derivatives (bottom).

FIG. 3. H2B serine 33 is phosphorylated in Drosophila. (A) Coomassie blue stained gel (top) and corresponding Western blot (bottom) of purified nucleosomes, H2BT, H2BT-S33P peptide (phosphorylated at S33), H3T peptide, H3T-S10/28P peptide (phosphorylated at S10 and S28), and recombinant H2B. Phosphorylation of H2B-S33 was monitored using the H2B-S33P antibody. (B) Western blots of in vitro kinase assays programmed with ATP, recombinant H2B, and TAF1 CTK, TBP, or TFIID and probed with the H2B-S33P antibody. (C) Coomassie blue stained gel (top) and corresponding Western blot (bottom) of nucleosomal histones purified from embryos and recombinant H2B and probed with the H2B-S33P antibody. (D) Coomassie blue stained gel (left) and corresponding Western blots (middle and right) of histone octamers purified from mock (+) or TAF1 (−) RNAi S2 cells. The same Western blot was probed with the H2B-S33P antibody, stripped, and reprobed with an H2B antibody as a loading control. (E) Western blots of whole cell extracts from mock (+) or TAF1 (−) RNAi S2 cells probed with antibodies to TAF1 (top) or SIN3 (bottom).

FIG. 4. Transcription activation in Drosophila coincides with TAF1-mediated phosphorylation of H2B-S33. (A) Photographs of ethidium bromide stained agarose gels showing RT-PCR products for stg (left) and actin5C (right) transcripts in mock (+) and TAF1 (−) RNAi S2 cells. (B) Photographs of ethidium bromide stained agarose gels showing PCR products for the stg promoter (P) or coding region (CR) in mock (+) and TAF1 (−) RNAi S2 cells. In vivo cross-linked chromatin was immunoprecipitated using the indicated antibodies or rabbit preimmune serum (control). Input represents the amount of stg promoter present in 0.1% of the chromatin used for XChIP (C) Gt transcription in heterozygous mutant cad (top), homozygous mutant TAF1^{CTK} (middle), or heterozygous mutant cad and homozygous mutant TAF1^{CTK} (bottom) blastoderm stage Drosophila embryos. Gt transcription was detected by in situ hybridization using gt anti-sense RNA. Anterior is to the left and dorsal is up. Arrows indicate the position of the posterior gt transcription domain. (D) Photographs of ethidium bromide stained agarose gels showing PCR products for the gt promoter (P) or coding region (CR) in embryos described in (C). In vivo cross-linked chromatin was isolated from the posterior halves of embryos and immunoprecipitated using the indicated antibodies or rabbit preimmune serum (control). Input represents the amount of gt promoter present in 2% of the chromatin used for XChIP.

FIG. 5. Protein sequence comparison of the serine/threonine kinase catalytic motif in the first bromodomain of TAF1 and other Drosophila protein kinases.

FIG. 6. (A) Silver stained gel corresponding to the autoradiograms shown inn FIG. 2B) of in vitro kinase assays containing γ³²P-ATP, native nucleosomes (top) or recombinant H2B (bottom), and TFIID, TAF1, or TAF1 derivatives (B) CTK(D1548A) binds acetylated histone H4. Western blot of protein:protein interaction assays. Anti-Flag antibody agarose beads loaded with recombinant Flag-CTK, Flag-CTK(D1548), or FLAG-110 (beads) were incubated with H4 peptide (amino acids 1-20) or H4 peptide [acetyl(H4)] acetylated at lysine 5, 8, 12, 16. Precipitated peptides were separated by SDS-PAGE, electrophoretically transferred onto nitrocellulose, and detected using antibodies to H4 (top) and acetylated H4 (bottom). (C) The CTK phosphorylates serine residues in H2BT. A schematic representation of H2BT is shown on top with highlighted positions of threonines (T) and serines (S). Autoradiograms (A) and corresponding coomassie blue stained gels (C) of in vitro kinase assays containing (+) or lacking (−) TAF1, γ³²P-ATP, and the indicated amount of H2BT or the following H2BT-derivatives: H2BT-ΔS (containing alanines instead of serines 5 and 33); H2BT-ΔT (containing alanines instead of threonines 4, 20, and 22); and H2BT-ΔST (containing alanines instead of all serine and threonine residues).

FIG. 7. Knockdown of TAF1 causes G2/M phase cell cycle arrest in Drosophila cells. Mock (left) and TAF1 (right) RNAi treated S2 cells were analyzed by flow cytometry. Phases of the cell cycle (G1, S, and G2/M) are indicated. On average, for asynchronously growing mock RNAi cells 40% of the cells were in G2/M while for TAF1 RNAi cells 83% of the cells were in G2/M.

FIG. 8. Schematic representation of Realtime PCR experiments detecting the string promoter (A) or the giant promoter (B) by XChIP. Chromatin was prepared from TAF1 RNAi and mock RNAi cells (A) or Drosophila embryos of the indicated genotype (B). To confirm the results shown in FIGS. 4B, 4D and 9, Realtime PCR was used to detect the stg and gt promoter in the same immunoprecipitated DNA pools that were used to generate the results shown in FIGS. 4 and 9. DNA was amplified in the presence of SYBR green. The comparative threshold cycle (C_{T}) method was used to compare the presence of the stg or gt promoter in immunoprecipitated DNA pools derived from TAF1, SIN3, or mock RNAi cells. (A) Schematic representation indicating the relative difference (in fold) of the indicated histone modifications at the stg promoter in mock RNAi cells compared to TAF1 RNAi cells (left panel) or vice versa (right panel). (B) Schematic representation of the relative amounts of precipitated gt promoter in DNA pools generated by XChIP using the indicated antibodies. Chromatin was prepared from embryos laid by cad/+ females expressing wild type TAF1 or TAF1ΔCTK or homozygous mutant TAF1^{CTK} embryos.

FIG. 9. (A) Histone modification pattern at the actin5C promoter. Photograph of an ethidium bromide stained agarose gel showing PCR products representing a 500 bp fragment containing the actin5C promoter in mock (+) and TAF1 (−) RNAi treated S2 cells. In vivo cross-linked and sonicated chromatin was immunoprecipitated using the indicated antibodies. Input represents the amount of actin5C promoter present in 0.1% of input chromatin. (B) Phosphorylation of H2B-S33 at the stg promoter in SIN3 RNAi cells. Photograph of an ethidium bromide stained agarose gel PCR products representing a 500 bp fragment containing the stg promoter in mock (+) and SIN3 (−) RNAi treated S2 cells. In vivo cross-linked and sonicated chromatin was immunoprecipitated using the indicated antibodies or rabbit preimmune serum as a control. Stg transcription is repressed in SIN3 RNAi treated S2 cells, which are arrested in the G2 phase of the cell cycle (18). (C) Histone modification pattern of the stg promoter in Drosophila cells. Photograph of an ethidium bromide stained agarose gel showing PCR products representing a 500 bp fragment containing the stg promoter in mock (+) and TAF1 (−) RNAi treated S2 cells. In vivo cross-linked and sonicated chromatin was immunoprecipitated using the indicated antibodies or rabbit preimmune serum as a control. Input represents the amount of stg promoter present in 0.1% of input chromatin.

FIG. 10. The mutant protein TAF1ΔCTK is expressed in Drosophila. (A) Schematic representation of TAF1 and epitopes recognized by TAF1 antibodies TAF1-M and TAF1-C. The position of the premature stop codon mutation in TAF1^{CTK} is indicated. (B) Western blot analysis using whole protein extracts generated from wild type Drosophila ovaries (wild type) or heterozygous mutant TAF1^{CTK}/+ ovaries. Extracts were separated by SDS-PAGE, electrophoretically transferred onto nitrocellulose and probed with TAF-C antibody (left) or TAF1-M antibody (right). Arrows and arrowhead indicate the positions of TAF1 and TAF1ΔCTK, respectively. (C) Western blot analysis detecting TAF1 and TAF1ΔCTK (top) or TBP (bottom) in nuclear extracts prepared from cad/+, TAF^{CTK}, and cad/+; TAF1^{CTK} embryos. TAF1 and TAF1ΔCTK were detected using the TAF1-C (left) or TAF1M antibody (right), respectively.

FIG. 11. Protein sequence comparison of the NH₂-terminal tails of H2B in various eukaryotic organisms. The position of H2B-S33 in Drosophila H2B and the corresponding serine (S) or threonine (T) residue in H2B's from other species are highlighted.

FIG. 12. Phosphorylation of H2B-S33 during the Drosophila cell cycle. Photograph of XChIP experiments detecting phosphorylated H2B-S33 and TAF1 at the string promoter in G2/M-, G1-, and S-phase of Drosophila S2 cells. Chromatin was prepared from 5×10⁶ sorted cells and immunoprecipitated using the indicated antibodies or rabbit pre-immune serum (control). PCR was used to detect a 500 bp genomic DNA fragment (genomic) containing the string promoter in precipitated DNA pools. Input represents 0.1% of the starting material.

FIG. 13. (A) The anti-H2B-S33P antibody detects phosphorylated serine 33 (Drosophila H2B)/36 (human H2B) in H2B in histone preparations from Drosophila, bovine and human HELA cells. (Left) Coomassie stained SDS-page polyacrylamide gel showing histones prepared from Drosophila, bovine calves, and HELA cells. The position of histones is indicated. (Right) Western Blot analysis of recombinant Drosophila H2B, and histone preparations from Drosophila, calf, and HELA cells. Proteins were separated by SDS-PAGE, transferred onto nitrocellulose, and detected using the antibody recognizing phopshorylated H2B-S33 (arrowhead). (B) Photographs of ethidium bromide stained agarose gel showing PCR products for the human cdc25 promoter in sorted wild type HELA cells (−) and HELA cells lacking TAF1 due to RNAi (+). In vivo cross-linked chromatin was isolated from G2/M, G1 and S phase cells and immunoprecipitated using the indicated antibodies or rabbit preimmune serum (control). Input represents the amount of gt promoter present in 2% of the chromatin used for XChIP.

FIG. 14. Alignment of Drosophila (D. mel) and human TAF1 C-terminal kinase domains. The TAF1 regions shown are: Drosophila TAF1 amino acids 1499-2132, and human TAF1: amino acids 1427-1893. Identical residues are indicated by tall, red bars, and residues that differ between the two sequences are indicated by short, blue bars. (A) First (amino-terminal) portion of sequence illustrated; (B) continuation of sequence illustrated.

FIG. 15. Alignment of Drosophila (D. mel) and human H3 proteins. Identical residues are indicated by tall, red bars, and residues that differ between the two sequences are indicated by short, blue bars.

FIG. 16. Monoclonal antibodies recognizing phosphorylated serine 33 in Drosophila histone H2B in vitro and in vivo. (A) Western blot of Drosophila H2BT (amino acids 1-39), H2BT-S33p peptide that is phosphorylated at S33, H3T peptide (amino acids amino acids 1-32), and H3T-S10p peptide that is phosphorylated at S10 and S28. Phosphorylation of H2B-S33 was monitored by Western blot using the rat monoclonal antibodies anti-H2B-S33p-A9 and anti-H2B-S33p-D7. The position of the immuno-reactive signal corresponding to H2BT-S33p is indicated (arrowhead). (B) Western blot of histone octamers purified from Drosophila S2 cells incubated with mock double-stranded RNA (mock) or double-stranded RNA targeting the transcript of TAF1 (TAF1 RNAi). The Western blot was probed with anti-H2B-S33p-A9 (left) and anti-H2B-S33p-D7 (right) antibodies, and an antibody recognizing ubiquitin as a loading control. The position H2B phosphorylated at serine 33 (H2B-S33p) and ubiquitin are indicated.

### DETAILED DESCRIPTION

The present invention provides a method of characterizing the proliferative state of cells in a biological sample based on detecting transcription factor-mediated phosphorylation of histone H2B (H2B) at residue 36 (numbered according to human H2B). This method optionally includes detection of transcription factor-mediated acetylation of histone H3 (H3) at residue 14 and/or acetylation of histone H4, which also provides an indication of cell proliferation. The invention also provides antibodies specific for H2B phosphorylated at H2B, which can be employed in the characterization method. Other aspects of the invention include transcription factor and H2B derivatives and related polynucleotides, vectors, host cells, recombinant production methods and compositions, as well as screening methods for modulators of H2B phosphorylation at residue 36.

Definitions

Terms used in the claims and specification are defined as set forth below unless otherwise specified.

As used herein, the term “biological sample” refers to any physiological medium containing histone H2B. A biological sample will generally also include histone H3, TAF1, and chromatin, preferably chromatin comprising one or more cell cycle genes, genes that participate in differentiation, and/or oncogenes. A biological sample can be obtained, for example, from cell culture or directly from an organism and may be subjected to any desired processing steps, e.g., concentration or dilution.

The following terms encompass polypeptides that are identified in Genbank by the following designations, as well as polypeptides that are at least about 70% identical to polypeptides identified in Genbank by these designations: TAF1, histone H2B, histone H3, H4, cdc25; Drosophila string; and Drosophila giant. In alternative embodiments, these terms encompass polypeptides identified in Genbank by these designations and polypeptides sharing at least about 80, 90, 95, 96, 97, 98, or 99% identity.

A “cell cycle gene” is any gene that encodes a protein that contributes to progression through any phase of the cell cycle. The term encompasses genes from any organism, but particularly those from eukaryotes, more particularly vertebrates, even more particularly mammals, and most particularly humans. Examples of cell cycle genes include: human cdc25 and Drosophila melanogaster string, which are both cell cycle division (Cdc) proteins; E2F transcription factors; mdm2; cyclins, cyclin-dependent kinases (CDKs); CDK inhibitors, Retinoblastoma (Rb)-associated proteins; and Constitutive Kinase Subunit.

A “gene that participates in tissue differentiation” is any gene that encodes a protein having a defined role in the differentiation of any tissue of any organism as well as any gene that is expressed in both a tissue- and developmental stage-specific manner. The term encompasses genes from any organism, but particularly those from eukaryotes, more particularly vertebrates, even more particularly mammals, and most particularly humans. The Drosophila giant gene, discussed in Example 1, is an exemplary gene that participates in tissue differentiation.

An “oncogene” is any gene that contributes to the formation of cancer when mutated or inappropriately (over_ or under-) expressed. Accordingly, as used herein, the term encompasses “proto-oncogenes.” Examples of oncogenes include: abl, ahi, akt, bcl, crk, dsi, erb, ets, evi, fes/fps, fim, fis, fgr, flv, fins, fos, gin, gli, int, jun, kit, mas, lck, met, mil/raf, mis, mlv, mos, myb, myc, neu, onc, pim, raf ras, Rb, rel, ros, seq, sis, ski, spi, src, tcl, thy, trk, and yes.

As used with respect to polypeptides or polynucleotides, the term “isolated” refers to a polypeptide or polynucleotide that has been separated from at least one other component that is typically present with the polypeptide or polynucleotide. Thus, a naturally occurring polypeptide is isolated if it has been purified away from at least one other component that occurs naturally with the polypeptide or polynucleotide. A recombinant polypeptide or polynucleotide is isolated if it has been purified away from at least one other component present when the polypeptide or polynucleotide is produced.

The terms “polypeptide” and “protein” are used interchangeably herein to refer a polymer of amino acids, and unless otherwise specified, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N. Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

As used with reference to a polypeptide, the term “full-length” refers to a polypeptide having the same length as the mature wild-type polypeptide.

The term “fragment” is used herein with reference to a polypeptide or a nucleic acid molecule to describe a portion of a larger molecule. Thus, a polypeptide fragment can lack an N-terminal portion of the larger molecule, a C-terminal portion, or both. Polypeptide fragments are also referred to herein as “peptides.” A fragment of a nucleic acid molecule can lack a 5′ portion of the larger molecule, a 3′ portion, or both. Nucleic acid fragments are also referred to herein as “oligonucleotides.” Oligonucleotides are relatively short nucleic acid molecules, generally shorter than 200 nucleotides, more particularly, shorter than 100 nucleotides, most particularly, shorter than 50 nucleotides. Typically, oligonucleotides are single-stranded DNA molecules.

A “subsequence” of an amino acid or nucleotide sequence is a portion of a larger sequence.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using a sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wisc.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Residues in two or more polypeptides are said to “correspond” if they are either homologous (i.e., occupying similar positions in either primary, secondary, or tertiary structure) or analogous (i.e., having the same or similar functional capacities). As is well known in the art, homologous residues can be determined by aligning the polypeptide sequences for maximum correspondence as described above.

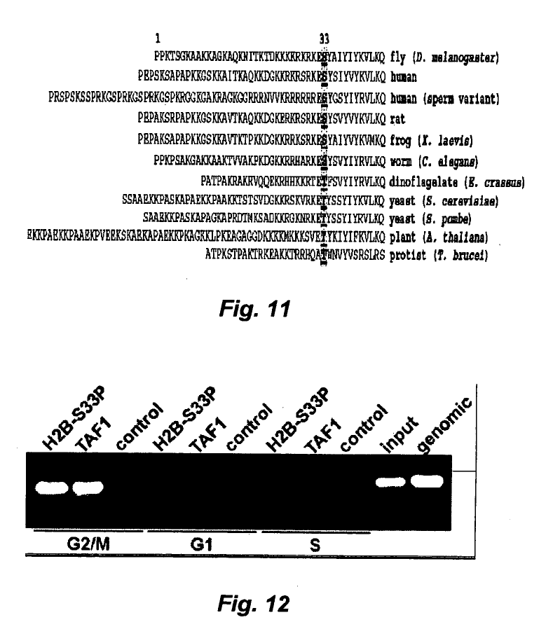

Residue positions in polypeptides discussed herein are identified with respect to a reference amino acid sequence. Thus, the phrase “histone H2B residues are numbered according to human H2B” indicates that the numbering of the human H2B protein is used to identify residue positions. In this case, a reference to “H2B residue 36” identifies a residue that, in human H2B, is the thirty-sixth amino acid from the N-terminus. This residue is a serine in human H2B. Those of skill in the art appreciate that this residue can have a different position in H2B proteins from different species and, indeed, can be a different amino acid, such as threonine. Thus, as used herein, “phosphorylation of histone H2B (H2B) at residue 36, wherein H2B residues are numbered according to human H2B” refers to phosphorylation of histone H2B from any species at the residue homologous to human H2B residue 36. Referring to FIG. 11, this residue is residue 33 in the Drosophila melanogaster sequence and residue 53 in the second-listed human sequence (the sperm variant).

As used with reference to polypeptides, the term “wild-type” refers to any polypeptide having an amino acid sequence present in a polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized.

The term “amino acid sequence variant” refers to a polypeptide having an amino acid sequence that differs from a wild-type amino acid sequence by the addition, deletion, or substitution of an amino acid.

The term “conservative amino acid substitution” is used herein to refer to the replacement of an amino acid with a functionally equivalent amino acid. Functionally equivalent amino acids are generally similar in size and/or character (e.g., charge or hydrophobicity) to the amino acids they replace. Amino acids of similar character can be grouped as follows:

(1) hydrophobic: His, Trp, Tyr, Phe, Met, Leu, Ile, Val, Ala;

(2) neutral hydrophobic: Cys, Ser, Thr;

(3) polar: Ser, Thr, Asn, Gln;

(4) acidic/negatively charged: Asp, Glu;

(5) charged: Asp, Glu, Arg, Lys, His;

(6) basic/positively charged: Arg, Lys, His;

(7) basic: Asn, Gln, His, Lys, Arg;

(8) residues that influence chain orientation: Gly, Pro; and

(9) aromatic: Trp, Tyr, Phe, His.

The following table shows exemplary and preferred conservative amino acid substitutions.

| | Exemplary Conservative | Preferred Conservative |
|---|---|---|
| Original Residue | Substitution | Substitution |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Asn |
| Ile | Leu, Val, Met, Ala, Phe | Leu |
| Leu | Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala | Leu |

As used with reference to a polypeptide or polypeptide fragment, the term “derivative” includes amino acid sequence variants as well as any other molecule that differs from a wild-type amino acid sequence by the addition, deletion, or substitution of one or more chemical groups. “Derivatives” retain at least one biological or immunological property of a wild-type polypeptide or polypeptide fragment, such as, for example, the biological property of specific binding to a receptor and the immunological property of specific binding to an antibody.

The term “polynucleotide” refers to a deoxyribonucleotide or ribonucleotide polymer, and unless otherwise specified, includes known analogs of natural nucleotides that can function in a similar manner to naturally occurring nucleotides.

The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA.

The term “polynucleotide” encompasses double-stranded polynucleotides, as well as single-stranded molecules. Double-stranded polynucleotides that encode a protein contain a “sense” polynucleotide strand hydrogen-bonded to an “antisense” polynucleotide strand. The sense polynucleotide strand is the strand whose nucleotide sequence, when translated, provides the amino acid sequence of the encoded protein. In double-stranded polynucleotides, the polynucleotide strands need not be coextensive (i.e., a double-stranded polynucleotide need not be double-stranded along the entire length of both strands).

As used herein, the term “complementary” refers to the capacity for precise pairing between two nucleotides, i.e. if a nucleotide at a given position of a nucleic acid molecule is capable of hydrogen bonding with a nucleotide of another nucleic acid molecule, then the two nucleic acid molecules are considered to be complementary to one another at that position.

The term “vector” is used herein to describe a construct, typically a DNA construct, containing a polynucleotide. Such a vector can be propagated stably or transiently in a host cell. The vector can, for example, be a plasmid, a viral vector, a cosmid, a BAC, a YAC, or simply a potential genomic insert. Once introduced into a suitable host, the vector may replicate and function independently of the host genome, or may, in some instances, integrate into the host genome.

“Expression vector” refers to a construct containing a polynucleotide molecule that is operably linked to a control sequence capable of effecting the expression of the polynucleotide in a suitable host. Exemplary control sequences include a promoter to effect transcription, an optional operator sequence to control transcription, a sequence encoding a suitable mRNA ribosome binding site, and sequences that control termination of transcription and translation.

As used herein, the term “operably linked” refers to a functional linkage between two sequences, such a control sequence (typically a promoter) and the linked sequence.

The term “host cell” refers to a cell capable of maintaining a vector either transiently or stably. Host cells of the invention include, but are not limited to, bacterial cells, yeast cells, insect cells, plant cells, and mammalian cells. Other host cells known in the art, or which become known, are also suitable for use in the invention.

A “normal” cell or tissue is one that is free of a particular pathology of interest. Thus, a normal cell can be “abnormal” in some respect, but still be a “normal” cell for the purposes of the invention.

The phrase “level of transcriptional activation” encompasses any level of transcriptional activation ranging from no transcription to a maximal transcription level for a particular gene. Thus, an “indication of the level of transcriptional activation” of a gene includes an indication that the gene is not being transcribed, as well as an indication that the gene is being transcribed (i.e., a qualitative indication that the gene is “off” or “on”). In addition, this phrase encompasses quantitative indications that transcription is occurring at a higher or lower rate in a test sample than in a control sample.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term “antibody”, as used herein also includes antibody fragments either produced by the modification of whole antibodies or synthesized de novo using recombinant DNA methodologies. Preferred antibodies include single chain antibodies, more preferably single chain Fv (scFv) antibodies in which a variable heavy and a variable light chain are joined together (directly or through a peptide linker) to form a continuous polypeptide.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs.” Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface.” This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

A single chain Fv (“scFv”) antibody is a covalently linked V_{H}::V_{L} heterodimer that forms a single antigen binding domain. Two scFv chains can be linked, covalently or noncovalently, to form an (scFv′)₂ antibody, which has two antigen binding domains, which can be the same or different.

As used herein, the term “antibody” includes any antibody conjugated to any other substance, e.g., labeled antibodies, antibodies conjugated to polymeric beads, etc.

As used herein, the terms “antibody binding” and “immunoreactivity” refer to the non-covalent interactions of the type which occur between an immunoglobulin molecule and an antigen for which the immunoglobulin is specific. The strength or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity and is thus equal to the dissociation constant K_{d}. See, generally, Davies et al. Ann. Rev. Biochem., 59: 439-473 (1990).

The phrase “specifically binds” is used herein with reference to an antibody to describe a binding reaction which is determinative of the presence of the corresponding antigen in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for a particular protein. For example, an anti-H2B-r36p antibody of the invention preferentially binds to H2B-r36p over the H2B form lacking a phosphate at on H2B residue 36. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

The term “anti-H2B-r36p antibody,” as used herein, refers to an antibody that specifically binds to H2B comprising a phosphate on H2B residue 36.

The term “in vivo cross-linked chromatin immunoprecipitation” refers to a technique whereby chromatin is cross-linked, generally by treating cells or tissue with a chemical agent (e.g., formamide) to preserve the association of protein and DNA in the chromatin structure. The resulting complex is typically sheared, followed by immunoprecipitation using an antibody specific for one of the proteins in the complex. Immuoprecipitation results in recovery of the DNA with which the protein of interest is complexed. This DNA can subsequently be analyzed (e.g., by polymerase chain reaction (PCR)) to identify the DNA sequence(s) associated with the protein of interest.

As used herein, an “indicator” of cell proliferation refers to any phenomenon that is associated with cell proliferation. An indicator of cell proliferation need not be dispositive, by itself. Rather, such an indicator can be used with one or more other factors to provide a definitive determination of proliferation state.

A “test agent” is any agent that can be screened in the prescreening or screening assays of the invention. The test agent can be any suitable composition, including a small molecule, peptide, or polypeptide.

Abbreviations

TFIID—Drosophila general transcription factor (GTF); a multi-protein complex including a TATA-box binding protein (TBP) and at least 12 TBP-associated factors (TAFs).

TAF1 (TAF_{II}250)—a subunit of TFIID that has kinase activity.

NTK—N-terminal kinase domain.

CTK—COOH-terminal kinase domain.

HAT—histone acetyltransferase domain.

DBD—double bromodomain.

XchIP—chromatin immunoprecipitation.

stg—Drosophila gene string.

gt—Drosophila gene giant.

TAF1-M—antibody that recognizes the middle of TAF1.

TAF1-C—antibody that recognizes the CTK of TAF1.

Detection of Phosphorylation of Histone H2B

The invention provides a method of characterizing the proliferative state of cells in a biological sample that entails detecting the phosphorylation of histone H2B (H2B) at residue 36, wherein H2B residues are numbered according to human H2B. Phosphorylation at H2B residue 36 is detected as an indicator of cell proliferation.

In preferred embodiments, the method additionally includes determining whether H2B that is phosphorylated at H2B residue 36 (H2B-r36p) is physically associated with the promoter of a gene. The physical association of H2B-r36p with a gene promoter provides an indication of the gene's level of transcriptional activation. In particular, the presence of H2B-r36p at a gene promoter is correlated with transcriptional activation.

The studies detailed herein demonstrate that the presence of H2B phosphorylated at residue 33 (which is the Drosophila residue corresponding to human residue 36) at the promoters of the Drosophila string (a homolog of the yeast Cdc25 cell cycle gene) and giant genes correlates with transcriptional activation of these genes. See Examples. A further study shows that H2B phosphorylated at residue 36 is present at the promoter of the bovine and human cdc25 genes. See Example 3.

The physical association of H2B-r36p with any gene of interest can be examined using the method of the invention. However, genes that play a role in cell proliferation and/or tissue differentiation are of particular interest because determining their level of transcriptional activation can be useful in the diagnosis and/or prognosis of cancer. Examples of such genes include cell cycle genes, illustrated herein using the human cdc25 gene and the D. melanogaster string gene. Of particular interest are genes encoding proteins that contribute to progression through the G2/M phase of the cell cycle, as the loss of the ability to phosphorylate H2B-r36 leads to G2/M arrest. Thus, H2B-r36 phorphorylation plays a particularly important role in regulating genes that are transcriptionally active during this phase of the cell cycle. See Examples.

Another class of genes that is amenable to analysis in the present method include more genes that participate in tissue differentiation, such as the Drosophila giant gene. As described in Example 1, H2B-S33P (serine 33 is the Drosophila residue corresponding to human residue 36) was detected at the transcriptionally active giant promoter in control embryos, but not at the transcriptionally repressed promoter in mutant embryos (cad/+; TAF³¹⁹⁴ ). The method of the invention can also be applied to oncogenes, such as abl, erb, myc, and the like.

Detection of phosphorylation of H2B residue 36 and, in preferred embodiments, H2B-r36p promoter association can be qualitative or quantitative, but is preferably quantitative. In a preferred embodiment, the amount of H2B-r36p physically associated with the promoter in a test sample is compared with the amount of H2B-r36p physically associated with the promoter in a control sample. The test and control samples can be any two samples that one wishes to compare. In general, the test sample is one that is suspected of containing proliferating cells that may, for example, indicate a pathological condition characterized by hyperproliferation of cells. Accordingly, the test sample can be a tissue biopsy taken from abnormal tissue, such as tissue suspected of being cancerous. In this instance, the control sample is preferably taken from normal tissue of the same type. The difference between the amount of H2B-r36p physically associated with the promoter in a test sample and the amount of H2B-r36p physically associated with the promoter in a control sample provides a metric useful in the diagnosis and/or prognosis of cancer.

In diagnostic/prognostic applications, H2B phosphorylation and, in preferred embodiments, promoter association need not be dispositive with respect to the existence of a particular pathology. Rather, the H2B phosphorylation level is used in the context of a differential diagnosis for that pathology. Accordingly, elevated H2B phosphorylation is used along with one or more other factors to provide a definitive diagnosis. In this context, H2B phosphorylation level is simply an indicator (one of many possible indicators) of a particular pathology (e.g., a particular tumor type or class, which may be associated with a particular outcome or responsive to one or more particular treatments).

Phosphorylation of H2B residue 36 can be detected in vivo or in vitro. However, phosphorylation assays of the invention are generally more conveniently carried out in vitro, for example, in a biological sample (e.g., whole blood, plasma, serum, saliva, synovial fluid, cerebrospinal fluid, bronchial lavage, ascites fluid, bone marrow aspirate, pleural effusion, urine, or tissue, cells, or fractions thereof) derived from an animal.

Although the sample is typically taken from a human, the assays can be used to detect H2B residue 36 phosphorylation in cells from any organism that produces this protein. Thus, the methods of the invention can be performed on samples from eukaryotes; particularly vertebrates; more particularly mammals, such as dogs, cats, sheep, cattle and pigs; and most particularly primates, such as humans, chimpanzees, gorillas, macaques, and baboons, and rodents such as mice, rats, and guinea pigs.

H2B residue 36 varies, depending on the species from which the sample is obtained. Thus, for example, human and rat H2Bs have a serine at residue 36, whereas yeast have a threonine at this position. The assay employed must therefore be capable of detecting phosphorylation of the residue homologous to human residue 36 of H2B from the species from which the sample is derived.

Tissue or fluid samples are obtained according to standard methods well known to those of skill in the art, such as, for example, by biopsy or venipuncture. The sample is optionally pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

Phosphorylation of histone H2B (H2B) at residue 36 can be detected and quantified by any of a number of other means well known to those of skill in the art. These can entail analytic biochemical methods such as spectrophotometry, radiography, electrophoresis, capillary electrophoresis, high performance liquid chromatography (HPLC), thin layer chromatography (TLC), hyperdiffusion chromatography, and the like, in addition to various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like. Also possible are non-immunological methods that employ a binding partner specific for H2B-r36p. Such a binding partner can be a natural or synthetic molecule that can be labeled and used in a manner similar to an antibody.

A. Immunoassays

In preferred embodiments, phosphorylation of histone H2B at residue 36 is detected using an immunoassay in which a biological sample comprising H2B is contacted with an antibody specific for H2B-r36p under conditions suitable for antibody binding, followed by detection of antibody binding. Such assays require an antibody that preferentially binds this phosphorylated form of H2B over the non-phosphorylated form or other proteins. Suitable antibodies are described in greater detail below.

H2B-r36p may be detected and optionally quantified using any of a number of well recognized immunological binding assays. (See for example, U.S. Pat. Nos. 4,366,241; 4,376,110; 4,517,288; and 4,837,168.) For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991)).

The immunoassays of the present invention can be performed in any of several configurations, e.g., those reviewed in Maggio (ed.) (1980) Enzyme Immunoassay CRC Press, Boca Raton, Fla.; Tijan (1985) “Practice and Theory of Enzyme Immunoassays,” Laboratory Techniques in Biochemistry and Molecular Biology, Elsevier Science Publishers B.V., Amsterdam; Harlow and Lane, supra; Chan (ed.) (1987) Immunoassay: A Practical Guide Academic Press, Orlando, Fla.; Price and Newman (eds.) (1991) Principles and Practice of Immunoassays Stockton Press, NY; and Ngo (ed.) (1988) Non isotopic Immunoassays Plenum Press, NY.

Immunoassays often utilize a labeling agent to specifically bind to and label the binding complex formed by the antibody and the analyte (i.e., an antibody/H2B-r36p complex). The labeling agent may itself be one of the moieties comprising the antibody/analyte complex. Thus, the labeling agent may be a labeled anti- H2B-r36p antibody. Alternatively, the labeling agent can be a third moiety, such as another antibody, that specifically binds to the anti-H2B-r36p antibody, the H2B-r36p, the antibody/ H2B-r36p complex, or to a group (e.g., biotin) that is covalently linked to the anti-H2B-r36p antibody or the H2B-r36p.

In one embodiment, the labeling agent is an antibody that specifically binds to the anti-H2B-r36p antibody. Such agents are well known to those of skill in the art, and most typically comprise labeled antibodies that specifically bind antibodies of the particular animal species from which the anti-H2B-r36p antibody is derived (e.g., an anti-species antibody). Thus, for example, where the anti-H2B-r36p antibody is a rabbit antibody, the labeling agent may be a mouse anti-rabbit IgG (i.e., an antibody specific to the constant region of the rabbit antibody.)

Other proteins capable of specifically binding immunoglobulin constant regions, such as streptococcal protein A or protein G can also used as the labeling agent. These proteins are normal constituents of the cell walls of streptococcal bacteria. They exhibit a strong non-immunogenic reactivity with immunoglobulin constant regions from a variety of species. See, generally Kronval, et al., (1973) J. Immunol., 111: 1401-1406, and Akerstrom, et al., (1985) J. Immunol., 135:2589-2542.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, volume of solution, concentrations, and the like. Usually, the assays are carried out at ambient temperature, although they can be conducted over a range of temperatures, such as 5° C. to 45° C.

1. Non Competitive Assay Formats

Immunoassays for detecting are preferably either competitive or noncompetitive. Noncompetitive immunoassays are assays in which the amount of captured analyte (in this case, H2B-r36p) is directly measured. In one preferred “sandwich” assay, for example, the capture agent (e.g., anti-H2B-r36p antibody) is bound directly or indirectly to a solid substrate, where it is immobilized. These immobilized anti-H2B-r36p antibodies capture H2B-r36p present in a sample. The H2B-r36p thus immobilized is then bound by a labeling agent, such as an anti-H2B-r36p antibody bearing a label. Alternatively, the second antibody may lack a label, but it may, in turn, be bound by a labeled third antibody specific for antibodies of the species from which the second antibody is derived. Free, labeled antibody is washed away, and the remaining bound labeled antibody is detected.

2. Competitive Assay Formats

In competitive assays, the amount of analyte (e.g., H2B-r36p) present in the sample is measured indirectly by measuring the amount of an added (exogenous) analyte displaced (or competed away) from a capture agent (e.g., anti-H2B-r36p antibody) by the analyte present in the sample. In one competitive assay, a known amount of H2B-r36p is added to a test sample with an unquantified amount of H2B-r36p, and the sample is contacted with the capture agent (e.g., anti-H2B-r36p antibody). The amount of added H2B-r36p that binds to the anti-H2B-r36p antibody is inversely proportional to the concentration of H2B-r36p present in the test sample.

As noted above, the anti-H2B-r36p antibody can be immobilized on a solid substrate to facilitate bound versus free separation. The amount of H2B-r36p bound to the anti-H2B-r36p antibody is determined either by measuring the amount of H2B-r36p present in an immune complex, or alternatively by measuring the amount of free (uncomplexed) H2B-r36p.

3. Western Blots

Western blot analysis and related methods can conveniently be used to detect and quantify the presence of H2B-r36p in a sample. The technique generally comprises separating sample products by gel electrophoresis on the basis of molecular weight, transferring the separated products to a suitable solid support, (such as a nitrocellulose filter, a nylon filter, or derivatized nylon filter), and incubating the sample with the antibodies that specifically bind H2B-r36p. These antibodies can be directly labeled or alternatively can be subsequently detected using labeled secondary antibodies that specifically bind to the anti-H2B-r36p antibody.

B. Substrates

As mentioned above, depending upon the assay, a component, such as an anti-H2B-r36p antibody, can be bound to a solid surface. Many methods for immobilizing biomolecules to a variety of solid surfaces are known in the art. For instance, the solid surface may be a membrane (e.g., nitrocellulose), a microtiter dish (e.g., PVC, polypropylene, or polystyrene), a test tube (glass or plastic), a dipstick (e.g. glass, PVC, polypropylene, polystyrene, latex, and the like), a microcentrifuge tube, or a glass, silica, plastic, metallic or polymer bead. The desired component may be covalently bound, or noncovalently attached through nonspecific bonding.

A wide variety of organic and inorganic polymers, both natural and synthetic may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials that may be employed, include paper, glasses, ceramics, metals, metalloids, semiconductive materials, cements or the like. In addition, substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes may be employed depending upon the nature of the system.

In preparing the surface, a plurality of different materials may be employed, e.g., as laminates, to obtain various properties. For example, protein coatings, such as gelatin can be used to avoid non-specific binding, simplify covalent conjugation, enhance signal detection or the like.

If covalent bonding between a component and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. See, for example, Immobilized Enzymes, Ichiro Chibata, Halsted Press, New York, 1978, and Cuatrecasas, (1970) J. Biol. Chem. 245 3059.

In addition to covalent bonding, various methods for noncovalently binding an assay component can be used. Noncovalent binding is typically nonspecific absorption of a component to the surface. Typically, the surface is blocked with a second component to prevent nonspecific binding of labeled assay components. Alternatively, the surface is designed such that it nonspecifically binds one component but does not significantly bind another. For example, a surface bearing a lectin such as concanavalin A will bind a carbohydrate-containing compound but not a labeled protein that lacks glycosylation. Various solid surfaces for use in noncovalent attachment of assay components are reviewed in U.S. Pat. Nos. 4,447,576 and 4,254,082.

C. Reduction of Non Specific Binding

One of skill will appreciate that it is often desirable to reduce non specific binding in immunoassays and during analyte purification. Where the assay involves H2B-r36p, anti-H2B-r36p antibody, or other capture agent immobilized on a solid substrate, it is desirable to minimize the amount of non-specific binding to the substrate. Means of reducing such non-specific binding are well known to those of skill in the art. Typically, this involves coating the substrate with a proteinaceous composition. In particular, protein compositions such as bovine serum albumin (BSA), nonfat powdered milk, and gelatin are widely used.

D. Detection

Detection of phosphorylation is carried out by any known method. The particular label or detectable group used in the assay is not a critical aspect of the invention. The detectable group can be any material having a detectable physical or chemical property. Such detectable labels have been well-developed in the field of immunoassays and, in general, any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., LacZ, CAT, horseradish peroxidase, alkaline phosphatase and others, commonly used as detectable enzymes, either as marker gene products or in an ELISA), and colorimetric labels such as colloidal gold or colored glass or plastic (e.g. polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

Components can also be conjugated directly to signal-generating compounds, e.g., by conjugation with an enzyme or fluorophore. Enzymes of interest as labels will primarily be hydrolases, particularly phosphatases, esterases and glycosidases, or oxitranscription factoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

### Detection of the Physical Association of H2B-r36p with a Promoter

Preferred embodiments of the invention include a determination as to whether H2B-r36p is physically associated with the promoter of a gene of interest. This determination is most conveniently carried out using in vivo cross-linked chromatin immunoprecipitation (XchIP). This technique is described below in reference 15 and illustrated in the Examples herein. Briefly, cells are incubated with cross-linker that cross-links the DNA and associated proteins present in chromatin. Any suitable cross-linker, such as formamide can be employed. Other crosslinkers suitable for carrying out in vivo crosslinking are described, e.g., in U.S. Pat. No. 5,770,736 (filed Jun. 23, 1998) and U.S. Pat. No. 6,008,211 (filed Dec. 28, 1999). In the Examples, cross-linking was achieved by treating cells with 1.8% formaldehyde for 15 min. Chromatin was then isolated from the cells by. In vivo cross-linked chromatin was sheared to a desired average fragment length, which facilitates handling of the chromatin and provides the desired degree of resolution (i.e., allowing one to conclude that a protein of interest is bound to a promoter of interest, rather than a neighboring promoter). In the Examples, herein, the chromatin was sheared to an average length of about 700 basepairs.

The sheared chromatin is then subjected to immunoprecipitation with an antibody of interest. The antibody is contacted with the chromatin under conditions suitable for antibody binding. An anti-H2B-r36p antibody, for example, binds to H2B-r36p in the chromatin. Immunoprecipitation results in the recovery of the H2B-r36p antibody complexed with the H2B-r36p protein, which is cross-linked to the DNA to which the H2B-r36p protein was bound in vivo. Immunoprecipitation can be carried out using any conventional techniques, such as affinity chromatography over a column that binds the antibody, such as e.g. a Protein A column.

Antibody/chromatin complexes can then be incubated with an RNase and a proteinase (e.g., Proteinase K) to remove RNA and proteins, respectively, followed by a suitable treatment to reverse the cross-links. Where formamide is used as the cross-linker, incubation at 65° C. for about 6 hours is sufficient to reverse the cross-links. The DNA is purified and used as a template for amplification.

Suitable amplification methods for use in the invention include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc. PCR products can be analyzed by gel electrophoresis using ethidium bromide containing agarose gels and detected by UV illumination.

If it is desirable to compare the level of one promoter in the immunoprecipitated chromatin with the level of another promoter, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

Detection of Acetylation of Histones H3 and/or H4

The invention also provides a method of characterizing the proliferative state of cells in a biological sample that entails detecting acetylation of histones. In particular, acetylation of histone H3 (H3) at residue 14 (wherein H3 residues are numbered according to human H3) and acetylation of histone H4 (H4) provide additional indicators of cell proliferation. Thus, phosphorylation of H2B at residue 36, acetylation of H3 at residue 14, and acetylation of H4 can be detected individually or in any combination as indicators of cell proliferation.

In preferred embodiments, the method additionally includes determining whether H3 that is acetylated at H3 residue 14 (H3-r14a) or acetylated H4 is physically associated with the promoter of a gene. The physical association of H3-r14a or acetylated H4 with a gene promoter provides an indication of the gene's level of transcriptional activation. In particular, the presence of H3-r14a or acetylated H4 at a gene promoter is correlated with transcriptional activation.

The studies detailed herein demonstrate that the presence of H3 acetylated at residue 14 and acetylated H4 at the promoter of the Drosophila string (a homolog of the yeast Cdc25 cell cycle gene) gene correlates with transcriptional activation of these genes. See Examples.

The physical association of H3-r14a or acetylated H4 with any gene of interest can be examined using the method of the invention. However, genes that play a role in cell proliferation and/or tissue differentiation are of particular interest because determining their level of transcriptional activation can be useful in the diagnosis and/or prognosis of cancer. Examples of such genes include cell cycle genes, illustrated herein using the human cdc25 gene and the Drosophila string gene. Of particular interest are genes encoding proteins that contribute to progression through the G1 or G2/M phases of the cell cycle.

Another class of genes that is amenable to analysis in the present method include more genes that participate in tissue differentiation, such as the Drosophila giant gene. The method of the invention can also be applied to oncogenes, such as abl, erb, myc, and the like.

Detection of H3 or H4 acetylation and, in preferred embodiments, promoter association can be qualitative or quantitative, but is preferably quantitative. In a preferred embodiment, the amount of H3-r14a or acetylated H4 physically associated with the promoter in a test sample is compared with the amount of H3-r14a or acetylated H4 physically associated with the promoter in a control sample. The test and control samples can be any two samples that one wishes to compare. In general, the test sample is one that is suspected of containing proliferating cells that may, for example, indicate a pathological condition characterized by hyperproliferation of cells. Accordingly, the test sample can be a tissue biopsy taken from abnormal tissue, such as tissue suspected of being cancerous. In this instance, the control sample is preferably taken from normal tissue of the same type. The difference between the amount of H3-r14a or acetylated H4 physically associated with the promoter in a test sample and the amount of H3-r14a or acetylated H4 physically associated with the promoter in a control sample provides a metric useful in the diagnosis and/or prognosis of cancer.

In diagnostic/prognostic applications, histone acetylation and, in preferred embodiments, promoter association need not be dispositive with respect to the existence of a particular pathology. Rather, the H3 or H4 acetylation level is used in the context of a differential diagnosis for that pathology. Accordingly, elevated H3 or H4 acetylation is used along with one or more other factors to provide a definitive diagnosis. In this context, H3 acetylation level is simply an indicator (one of many possible indicators) of a particular pathology (e.g., a particular tumor type or class, which may be associated with a particular outcome or responsive to one or more particular treatments).

Histone acetylation and promoter association can be determined as described above for histone phosphorylation and as illustrated in Example 1. Antibodies specific for H3-r14a or acetylated H4 are known and can be produced according to the methods described here.

Identification of Eukaryotic Promoters

Another aspect of the invention is a method for identifying one or more eukaryotic promoters in a biological sample, based on physical association in vivo with histone H2B (H2B) that is phosphorylated at H2B residue 36 (H2B-r36p). The method entails isolating from the sample one or more polynucleotides that are physically associated in vivo with H2B-r36p. H2B residues are numbered according to human H2B. Although any suitable technique can be employed, the isolation of polynucleotides that are physically associated in vivo with H2B-r36p is preferably carried out by in vivo cross-linked chromatin immunoprecipitation, as described above. This method can be carried out using any convenient sample from any species that expresses H2B, as described above for the other methods of the invention.

In vivo cross-linked chromatin immunoprecipitation will, in most embodiments, yield a plurality of different polynucleotides. The precipitated polynucleotides can be further analyzed by any conventional method for analyzing polynucleotides. The precipitated polynucleotides can be analyzed directly or amplified as described above and the amplification product further analyzed. In preferred embodiments, the precipitated polynucleotides or amplification products are further analyzed by hybridization to one or more known polynucleotides. Hybridization indicates that the known polynucleotide sequence was physically associated with H2B-r36p and is therefore likely to include promoter sequences.

Array-Based Hybridization

Hybridization analysis of the precipitated polypeptides or amplification products is most conveniently carried out by DNA microarray analysis. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly polynucleotide arrays can be produced according to a wide variety of methods well known to those of skill in the art. For example, in a simple embodiment, “low density” arrays can simply be produced by spotting (e.g., by hand using a pipette) different polynucleotides at different locations on a solid support (e.g., a glass surface, a membrane, etc.). This simple spotting, approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide microarrays. Synthesis of high density arrays is also described in U.S. Pat. Nos. 5,744,305; 5,800,992; and 5,445,934.

In one embodiment, the arrays used in this invention are arrays of “probe” polynucleotides. These probes are then hybridized respectively with their “target” polynucleotides. The arrays can be hybridized with a single population of sample polynucleotide or can be used with two differentially labeled collections (as with a test sample and a reference sample).

Many methods for immobilizing polynucleotides on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, and the like. In addition, substances that form gels can be used. Such materials include, e.g., proteins (e.g., gelatins), lipopolysaccharides, silicates, agarose, and polyacrylamides.

In preparing the surface, any of a variety of different materials may be employed, particularly as laminates, to provide desirable properties. For example, proteins (e.g., bovine serum albumin) or mixtures of macromolecules (e.g., Denhardt's solution) can be employed to reduce non-specific binding, simplify covalent conjugation, or enhance signal detection. If covalent bonding between a compound and the surface is desired, the surface will usually be polyfunctional or be capable of being polyfunctionalized. Functional groups which may be present on the surface and used for linking polynucleotides can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. For example, polynucleotides can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). In addition, polynucleotides are conveniently modified by introduction of various functional groups that facilitate immobilization (see, e.g., Bischoff (1987) Anal. Biochem., 164: 336-344; Kremsky (1987) Nucl. Acids Res. 15: 2891-2910).

Arrays can be made up of target elements of various sizes, ranging from 1 mm diameter down to 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Hybridization Conditions

Polynucleotide hybridization simply involves providing a denatured probe and target polynucleotide under conditions where the probe and its complementary target can form stable hybrid duplexes through complementary base pairing. The polynucleotides that do not form hybrid duplexes are then washed away leaving the hybridized polynucleotides to be detected, typically through detection of an attached detectable label. Polynucleotides are generally denatured by increasing the temperature or decreasing the salt concentration of the buffer containing the polynucleotides, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In general, there is a tradeoff between hybridization specificity (stringency) and signal intensity. In a preferred embodiment, the wash is performed at the highest stringency that produces consistent results and that provides a signal intensity greater than approximately 10% of the background intensity. Hybridization can performed at low stringency to ensure hybridization and then subsequent washes are performed to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be included in the reaction mixture.

Methods of optimizing hybridization conditions are well known to those of skill in the art (see, e.g., Tijssen (1993) Laboratory Techniques in Biochemistry and Molecular Biology, Vol. 24: Hybridization With Polynucleotide Probes, Elsevier, N.Y.). In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Detection

In a preferred embodiment, the hybridized polynucleotides are detected by detecting one or more labels attached to the sample polynucleotides. Detectable labels suitable for use in the present invention include any composition detectable by spectroscopic, photochemical, biochemical, immunochemical, electrical, optical or chemical means. Exemplary labels are listed above in the “Immunoassay” section.

The label may be added to the target (sample) polynucleotide(s) prior to, or after the hybridization. So-called “direct labels” are detectable labels that are directly attached to or incorporated into polynucleotide probes prior to hybridization. In contrast, so-called “indirect labels” typically bind to the hybrid duplex after hybridization. Often, the indirect label binds to a moiety that is attached to or incorporated into the polynucleotide probe prior to the hybridization. Thus, for example, the polynucleotide probe may be biotinylated before the hybridization. After hybridization, an avidin-conjugated fluorophore will bind the biotin bearing hybrid duplexes providing a label that is easily detected. For a detailed review of methods of labeling polynucleotides and detecting labeled hybridized polynucleotides see Laboratory Techniques in Biochemistry and Molecular Biology, Vol. 24: Hybridization With Polynucleotide Probes, P. Tijssen, ed. Elsevier, N.Y. (1993).

The labels may be incorporated by any of a number of means well known to those of skill in the art. Means of attaching labels to polynucleotides include, for example nick translation or end-labeling.

Antibodies Specific for Histone H2B Phosphorylated at Residue 36

The invention provides an antibody specific for histone H2B (H2B) comprising a phosphate on H2B residue 36, wherein H2B residues are numbered according to human H2B. The invention encompasses antibodies that are specific for H2B-r36p from any species, e.g., any eukaryote, and particularly vertebrate. However, antibodies specific for H2B-r36p from mammals are preferred and those specific for human H2B-r36p are more preferred. As residue 36 is either serine or threonine, depending on the species, antibodies that recognize H2B phosphorylated at serine 36 or threonine 36 are within the scope of the invention. The antibodies can be specific for H2B-r36p from one species or can cross-react with H2B-r36p from multiple species. Antibodies of the invention preferentially bind H2B comprising a phosphate on H2B residue 36 over the form lacking a phosphate at this residue or other proteins. The degree of preferential binding should be sufficient to allow use of the antibody to distinguish H2B-r36p from the form that is not phorphorylated at this residue under normal assay conditions, such as those described herein. The binding preference (e.g., affinity) for the H2B-r36p is generally at least about 2-fold, more preferably at least about 5-fold, and most preferably at least about 10-, 20-, 50-, 10²-, 10³-, 10⁴, 10⁵, or 10⁶-fold over a non-specific target molecule (e.g. a randomly generated molecule lacking the specifically recognized site(s)). Antibodies of the invention preferably have a binding affinity of about 10⁻⁶, about 10⁻⁷, about 10⁻⁸ or better.

The invention encompasses polyclonal and monoclonal anti-H2B-r36p antibodies. Polyclonal antibodies are raised by injecting (e.g. subcutaneous or intramuscular injection) antigenic polypeptides into a suitable mammal (e.g., a mouse or a rabbit). Generally, the polypeptide used to raise anti-H2B-r36p should induce production of high titers of antibody with relatively high affinity for H2B-r36p.

If desired, the immunizing polypeptide may be conjugated to a carrier protein by conjugation using techniques that are well known in the art. Commonly used carriers include keyhole limpet hemocyanin (KLH), thyroglobulin, bovine serum albumin (BSA), and tetanus toxoid. The conjugate is then used to immunize the animal.

The antibodies are then obtained from blood samples taken from the animal. The techniques used to produce polyclonal antibodies are extensively described in the literature (see, e.g., Methods of Enzymology, “Production of Antisera With Small Doses of Immunogen: Multiple Intradermal Injections,” Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal, as well as monoclonal, antibodies see, for example, Coligan, et al. (1991) Unit 9, Current Protocols in Immunology, Wiley Interscience).

In preferred embodiments, polyclonal anti-H2B-r36p antibodies are subjected to two affinity purification steps. First, the polyclonal antiserum is contacted with an H2B-r36p polypeptide, and the fraction that binds is recovered. This fraction is then contacted with an H2B polypeptide that is not phosphorylated at residue 36, and the fraction that fails to bind to the unphosphorylated form is recovered to obtain a polyclonal antiserum that is specific for H2B-r36p. See Examples.

For many applications, monoclonal anti-H2B-r36p antibodies are preferred. The general method used for production of hybridomas secreting mAbs is well known (Kohler and Milstein (1975) Nature, 256:495). Briefly, as described by Kohler and Milstein, the technique entailed isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody that bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

It is also possible to evaluate a mAb to determine whether it has the same specificity as a mAb described herein without undue experimentation by determining whether the mAb being tested prevents the described mAb from binding a target polypeptide. If the mAb being tested competes with the mAb described herein, it is likely that the two monoclonal antibodies bind to the same or a closely related epitope. Still another way to determine whether a mAb has the specificity of a mAb described herein is to preincubate the mAb described herein with an antigen with which it is normally reactive, and determine if the mAb being tested is inhibited in its ability to bind the antigen. Such inhibition indicates that the mAb being tested has the same, or a closely related, epitopic specificity as the mAb described herein.

The antibodies of the invention include single chain Fv (“scFv”) polypeptides, which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. There are a number of structures for converting the naturally aggregated, but chemically separated, light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

Design criteria include determination of the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Pat. Nos. 5,091,513 and 5,132,405 to Huston et al.; and U.S. Pat. No. 4,946,778 to Ladner et al.

In this regard, the first general step of linker design involves identification of plausible sites to be linked. Appropriate linkage sites on each of the V_{H} and V_{L} polypeptide domains include those that will result in the minimum loss of residues from the polypeptide domains, and that will necessitate a linker comprising a minimum number of residues consistent with the need for molecule stability. A pair of sites defines a “gap” to be linked. Linkers connecting the C-terminus of one domain to the N-terminus of the next generally comprise hydrophilic amino acids which assume an unstructured configuration in physiological solutions and preferably are free of residues having large side groups which might interfere with proper folding of the V_{H} and V_{L} chains. Thus, suitable linkers under the invention generally comprise polypeptide chains of alternating sets of glycine and serine residues and may include glutamic acid and lysine residues inserted to enhance solubility. One particular linker under the invention has the amino acid sequence [(Gly)₄Ser]₃. Another particularly preferred linker has the amino acid sequence comprising 2 or 3 repeats of [(Ser)₄Gly] such as [(Ser)₄Gly]₃. Nucleotide sequences encoding such linker moieties can be readily provided using various oligonucleotide synthesis techniques known in the art.

Single chain antibodies (scFv or others), can be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20-fold-1,000,000-fold are obtained for a single round of affinity selection. By infecting bacteria with the eluted phage, however, more phage can be grown and subjected to another round of selection. In this way, an enrichment of 1000-fold in one round can become 1,000,000-fold in two rounds of selection (McCafferty et al. (1990) Nature, 348: 552-554). Thus, even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Monovalent scFv antibodies can be converted to bivalent (scFv′)2 antibodies, in which two scFv chains are linked covalently or noncovalently. For example, de Kruif, J. and Logtenberg, T. (1996) J. Biol. Chem. 271:7630-4 describes the construction of leucine zipper-based dimerization cassettes for the conversion of recombinant scFv antibodies to (scFv′)2 antibodies. A truncated murine IgG3 hinge region and a Fos or Jun leucine zipper were cloned into four scFv fragments. Cysteine residues flanking the zipper region were introduced to covalently link dimerized scFv fragments. The secreted fusion proteins were shown to form stable Fos-Fos or Jun-Jun homodimers.

Human antibodies can be produced without prior immunization by displaying very large and diverse V-gene repertoires on phage (Marks et al. (1991) J. Mol. Biol. 222: 581-597). In one embodiment natural VH and VL repertoires present in human peripheral blood lymphocytes are isolated from unimmunized donors by PCR. The V-gene repertoires were spliced together at random using PCR to create a scFv gene repertoire which was cloned into a phage vector to create a library of 30 million phage antibodies. From this single “naïve” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1 nM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

As those of skill in the art readily appreciate, antibodies can be prepared by any of a number of commercial services (e.g., Berkeley antibody laboratories, Bethyl Laboratories, Anawa, Eurogenetec, etc.).

Kits

In another embodiment, the invention provides a kit useful for the detection of phosphorylation of H2B at residue 36. Kits will typically comprise one or more anti-H2B-r36p antibodies of this invention. For diagnostic purposes, the antibody(s) can be labeled. In addition, the kits will typically include instructional materials for carrying out any of the methods described herein. While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media can include addresses to internet sites that provide such instructional materials.

The kits may also include additional components to facilitate the particular application for which the kit is designed. Thus, for example, where a kit contains an anti-H2B-r36p antibody that is labeled, the kit may additionally contain means of detecting the label (e.g., enzyme substrates for enzymatic labels, filter sets to detect fluorescent labels, appropriate secondary labels, or the like). The kits may additionally include buffers and other reagents routinely used for the practice of a particular method. An exemplary kit useful in an immunoassay to detect phosphorylation of H2B at residue 36 include, in addition to an anti-H2B-r36p antibody, an H2B peptide or polypeptide that includes a phosphate at residue 36. This peptide or polypeptide can be employed, for example, as a positive control or as competitor in a competitive immunoassay, and can be labelled or not, depending on the format of the assay to be carried out.

In one embodiment, kit facilitates the detection of acetylation of H3 and/or H4, as well as the detection of phosphorylation of H2B at residue 36. Such kits will typically comprise one or more anti-H2B-r36p antibodies of this invention and one or more antibodies specific for acetylated H3 and/or acetylated H4. The anti-acetylated H3 antibody is preferably specific for H3 comprising an acetyl group on H3 residue 14, wherein H3 residues are numbered according to human H3. For diagnostic purposes, the antibody(s) can be labeled. An exemplary kit useful in an immunoassay to detect phosphorylation of H2B and acetylation of H3 and/or H4 includes, in addition to the appropriate antibodies, an H3 peptide or polypeptide that includes an acetyl group at residue 14 and/or an acetylated H4 peptide or polypeptide. This peptide or polypeptide can be employed, for example, as a positive control or as competitor in a competitive immunoassay, and can be labelled or not, depending on the format of the assay to be carried out.

Polypeptides

A. Transcription Factor Derivative

In one embodiment, the invention provides a polypeptide that is a derivative of a transcription factor. A transcription factor polypeptide of the invention includes a fragment of a transcription factor, wherein the fragment includes a double bromodomain kinase and phosphorylates histone H2B (H2B) at H2B residue 36, wherein H2B residues are numbered according to human H2B. As the amino acid at position 36 of H2B varies with species, the fragment is capable of phosphorylating a serine or a threonine at this position or either. In preferred embodiments, the transcription factor fragment includes a serine/threonine kinase domain and a double ATP binding motif. Transcription factor polypeptides of the invention do not generally contain more than about 650 contiguous amino acids of the transcription factor. In preferred embodiments, transcription factor polypeptides of the invention contain about 250, 300, 350, 400, 450, 500, or 550 contiguous amino acids of the transcription factor or have a number of contiguous amino acids that falls in a range between any of these values.

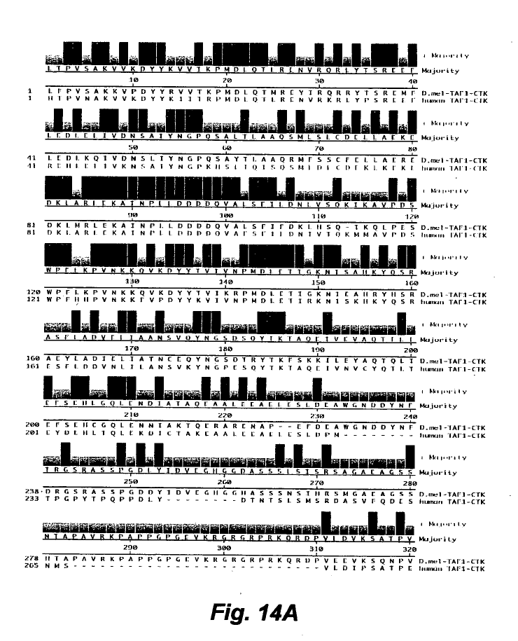

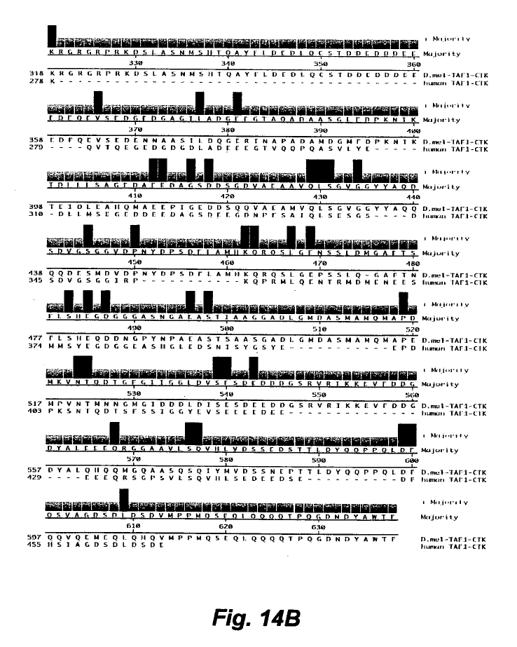

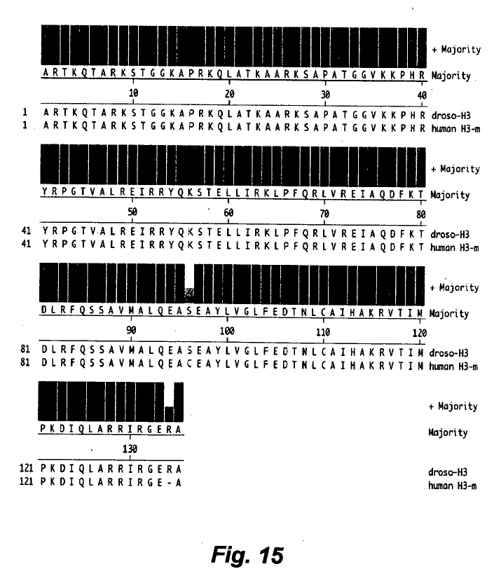

The transcription factor amino acid sequence can be derived from any transcription factor having the requisite kinase activity from any organism. Transcription factor amino acid sequences useful in the invention are generally derived from eukaryotes; particularly vertebrates; more particularly mammals, such as dogs, cats, sheep, cattle and pigs; and most particularly primates, such as humans, chimpanzees, gorillas, macaques, and baboons, and rodents such as mice, rats, and guinea pigs. In preferred embodiments, the transcription factor amino acid sequence is derived from a human transcription factor, such as human TAF1. An exemplary human transcription factor amino acid sequence, which includes amino acids 1427-1893 of human TAF1 is shown in FIG. 14. An exemplary transcription factor polypeptide from Drosophila is described in Example 1.

The transcription factor amino acid sequence can be a wild-type amino acid sequence or an amino acid sequence variant of the corresponding wild-type amino acid sequence. Preferred transcription factor polypeptides generally include a wild-type transcription factor amino acid sequence or a transcription factor amino acid sequence containing one or more conservative amino acid substitutions.

In addition to the amino acid sequences described above, transcription factor polypeptides of the invention can include other amino acid sequences, including those from heterologous proteins. Accordingly, the invention encompasses fusion polypeptides in which the above-discussed amino acid sequence is fused, at either or both ends, to amino acid sequence(s) from one or more heterologous proteins. Examples of additional amino acid sequences often incorporated into proteins of interest include a signal sequence, which facilitates purification of the protein, and an epitope tag, which can be used for immunological detection or affinity purification.

Transcription factor polypeptides of the invention can be otherwise modified to produce derivatives that retain the above-described functions, namely the ability to phosphorylate histone H2B (H2B) at H2B residue 36. In preferred embodiments, the modified polypeptides have an activity that is about 0.1 to about 0.01-fold that of the unmodified forms. In more preferred embodiments, the modified polypeptides have an activity that is about 0.1 to about 1-fold that of the unmodified polypeptides. In even more preferred embodiments, the modified polypeptides have an activity that is greater than the unmodified polypeptides.

Transcription factor polypeptides of the invention can be produced by any available technique, such as the synthetic and recombinant techniques discussed below.

B. Histone H2B Derivative

In one embodiment, the invention provides a polypeptide that is a derivative of a histone H2B. A H2B polypeptide of the invention includes a fragment of a histone H2B that includes H2B residue 36, the H2B residues being numbered according to human H2B. The amino acid at position 36 of the H2B fragment can be either a serine or a threonine. The H2B fragment generally retains the ability to bind to an antibody that cross-reacts with full-length H2B, and preferably retains the ability to bind to an epitope that includes H2B residue 36. This amino acid may be phosphorylated or not, depending on the intended use of the H2B polypeptide. For example, if the H2B polypeptide is to be used as a positive control in an immunoassay using an anti-H2B-r36p antibody of the invention, H2B-r36 will be phosphorylated, and the H2B polypeptide will retain the ability to bind to this antibody. The unphosphorylated form of this H2B polypeptide is useful, e.g., as a negative control in such an assay.

H2B polypeptides of the invention do not generally contain more than about 40 contiguous amino acids of the H2B. In preferred embodiments, H2B polypeptides of the invention contain about 5, 10, 15, 20, 25, 30, or 35 contiguous amino acids of H2B or have a number of contiguous amino acids that falls in a range between any of these values.

The H2B amino acid sequence can be derived from any H2B from any organism. H2B amino acid sequences useful in the invention are generally derived from eukaryotes; particularly vertebrates; more particularly mammals, such as dogs, cats, sheep, cattle and pigs; and most particularly primates, such as humans, chimpanzees, gorillas, macaques, and baboons, and rodents such as mice, rats, and guinea pigs. In preferred embodiments, the H2B amino acid sequence is derived from human H2B and includes the amino acid sequence SRKESYSIY (SEQ ID NO:______). An exemplary H2B polypeptide from Drosophila, which includes the amino acid sequence KRKESYAIY (SEQ ID NO:______) is described in Example 1.

The H2B amino acid sequence can be a wild-type amino acid sequence or an amino acid sequence variant of the corresponding wild-type amino acid sequence. Preferred H2B polypeptides generally include a wild-type H2B amino acid sequence or a H2B amino acid sequence containing one or more conservative amino acid substitutions.

In addition to the amino acid sequences described above, H2B polypeptides of the invention can include other amino acid sequences, including those from heterologous proteins. Accordingly, the invention encompasses fusion polypeptides, such as, for example, fusions including a signal sequence and/or an epitope tag.

H2B polypeptides of the invention can be otherwise modified to produce derivatives that retain the above-described immunoreactivity. In preferred embodiments, the modified polypeptides have an immunoreactivity that is about 0.1 to about 0.01-fold that of the unmodified forms. In more preferred embodiments, the modified polypeptides have an immunoreactivity that is about 0.1 to about 1-fold that of the unmodified polypeptides. In even more preferred embodiments, the modified polypeptides have an immunoreactivity that is greater than the unmodified polypeptides.

H2B polypeptides of the invention can be produced by any available technique, such as the synthetic and recombinant techniques discussed below.

C. Production of Polypeptides

1. Synthetic Techniques

H2B-r36p polypeptides according to the invention can be synthesized using methods known in the art, such as for example exclusive solid phase synthesis, partial solid phase synthesis, fragment condensation, and classical solution synthesis. See, e.g., Merrifield, J. Am. Chem. Soc., 85:2149 (1963). Solid phase techniques are preferred. On solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the tradename BIO-BEADS SX-1 by Bio Rad Laboratories, Richmond, Calif., and the preparation of the hydroxymethyl resin is described by Bodonszky, et al., Chem. Ind. (London), 38:1597 (1966). The benzhydrylamine (BHA) resin has been described by Pietta and Marshall, Chem. Commn., 650 (1970) and is commercially available from Beckman Instruments, Inc., Palo Alto, Calif., in the hydrochloride form. Automated peptide synthesizers are commercially available, as are services that make peptides to order.

Thus, the polypeptides of the invention can be prepared by coupling an alpha-amino protected amino acid to the chloromethylated resin with the aid of, for example, cesium bicarbonate catalyst, according to the method described by Gisin, Helv. Chim. Acta., 56:1467 (1973). After the initial coupling, the alpha-amino protecting group is removed by a choice of reagents including trifluoroacetic acid (TFA) or hydrochloric acid (HCl) solutions in organic solvents at room temperature.

Suitable alpha-amino protecting groups include those known to be useful in the art of stepwise synthesis of peptides. Examples of alpha-amino protecting groups are: acyl type protecting groups (e.g., formyl, trifluoroacetyl, acetyl), aromatic urethane type protecting groups (e.g. benzyloxycarboyl (Cbz) and substituted Cbz), aliphatic urethane protecting groups (e.g., t-butyloxycarbonyl (Boc), isopropyloxycarbonyl, cyclohexyloxycarbonyl), and alkyl type protecting groups (e.g., benzyl, triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride, dimethyl formamide (DMF) mixtures.

After the desired amino acid sequence has been completed, the desired peptide is decoupled from the resin support by treatment with a reagent such as trifluoroacetic acid or hydrogen fluoride (HF), which not only cleaves the peptide from the resin, but also cleaves all remaining side chain protecting groups. When the chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These and other solid phase peptide synthesis procedures are well known in the art and further described by John Morrow Stewart and Janis Dillaha Young, Solid Phase Peptide Syntheses (2nd Ed., Pierce Chemical Company, 1984).

2. Recombinant TechniQues

Polypeptides according to the invention can also produced using recombinant techniques. Precursor genes or gene sequences can be cloned, for instance, based on homology to the polypeptides described herein. With a precursor gene in hand, a nucleic acid molecule encoding a desired polypeptide can be generated by any of a variety of mutagenesis techniques. See, e.g., Sambrook, J., Fritsch, E. F., and Maniatis, T. (1989) in Molecular Cloning: A Laboratory Manual, Second Edition, Cold Spring Harbor, N.Y. Examples include site-specific mutagenesis (Kunkel et al., (1991) Methods Enzymol., 204:125-139; Carter, P., et al., (1986) Nucl. Acids Res. 10:6487), cassette mutagenesis (Wells, J. A., et al., (1985) Gene 34:315), and restriction selection mutagenesis (Wells, J. A., et al., (1986) Philos. Trans. R. Soc., London Ser. A, 317:415).

In a preferred embodiment of the invention, the sequence of a desired polypeptide is used as a guide to design a synthetic nucleic acid molecule. Methods for constructing synthetic genes are well known to those of skill in the art. See, e.g., Dennis, M. S., Carter, P. and Lazarus, R. A. (1993) Proteins: Struct. Funct. Genet., 15:312-321. Expression and purification methods are described below in connection with the nucleic acids, vectors and host cells of the invention.

Polynucleotides, Vectors, and Host Cells

The invention also provides polynucleotides encoding the polypeptides of the invention, vectors including these polynucleotides, and a host cells including the vectors. In one embodiment, the polynucleotide encodes a fragment of a transcription factor. The fragment includes a double bromodomain kinase and phosphorylates histone H2B (H2B) at H2B residue 36, wherein H2B residues are numbered according to human H2B. The transcription factor polynucleotide does not contain more than about 1950 contiguous nucleotides of transcription factor coding sequence. In preferred embodiments, transcription factor polynucleotides of the invention contain about 750, 900, 1050, 1200, 1350, 1500, or 1650 contiguous nucleotides of transcription factor coding sequence or have a number of contiguous nucleotides that falls in a range between any of these values.

In another embodiment, the polynucleotide encodes a fragment of an H2B. The fragment includes H2B residue 36, wherein the H2B residues are numbered according to human H2B. The amino acid at position 36 of the H2B fragment can be either a serine or a threonine. The H2B fragment generally retains the ability to bind to an antibody that cross-reacts with full-length H2B, and preferably retains the ability to bind to an epitope that includes H2B residue 36. The H2B polynucleotide generally does not comprise more than about 120 contiguous nucleotides of histone H2B coding sequence. In preferred embodiments, H2B polynucleotides of the invention contain about 15, 30, 45, 60, 75, 90, or 105 contiguous nucleotides of H2B coding sequence or have a number of contiguous nucleotides that falls in a range between any of these values.

A. Polynucleotides

Polynucleotides of the invention include a portion that encodes a transcription factor or H2B polypeptide. As noted above, the encoded amino acid sequence can be a wild-type sequence or a variant sequence. Where the amino acid sequence is a wild-type sequence, the nucleotide sequence encoding this sequence can be a wild-type nucleotide sequence or one containing “silent” mutations that do not alter the amino acid sequence due to the degeneracy of the genetic code. For example, if the polynucleotide is intended for use in expressing the encoding polypeptide, silent mutations can be introduced by standard mutagenesis techniques to optimize codons to those preferred by the host cell.

In some applications, it is advantageous to stabilize the polynucleotides described herein or to produce polynucleotides that are modified to better adapt them for particular applications. To this end, the polynucleotides of the invention can contain phosphorothioates, phosphotriesters, methyl phosphonates, short chain alkyl or cycloalkyl intersugar linkages or short chain heteroatomic or heterocyclic intersugar (“backbone”) linkages. Most preferred are phosphorothioates and those with CH2—NH—O—CH2, CH2—N(CH3)—O—CH2 (known as the methylene(methylimino) or MMI backbone) and CH2—O—N(CH3)—CH2, CH2—N(CH3)—N(CH3)—CH2, and O—N(CH3)—CH2—CH backbones (where phosphodiester is O—P—O—CH2). Also preferred are polynucleotides having morpholino backbone structures. Summerton, J. E. and Weller, D. D., U.S. Pat. No. 5,034,506. Other preferred embodiments use a protein-nucleic acid or peptide-nucleic acid (PNA) backbone, wherein the phosphodiester backbone of the polynucleotide is replaced with a polyamide backbone, the bases being bound directly or indirectly to the aza nitrogen atoms of the polyamide backbone. P. E. Nielsen, M. Egholm, R. H. Berg, O. Buchardt, Science 1991, 254, 1497. Polynucleotides of the invention can contain alkyl and halogen-substituted sugar moieties and/or can have sugar mimetics such as cyclobutyls in place of the pentofuranosyl group. In other preferred embodiments, the polynucleotides can include at least one modified base form or “universal base” such as inosine. Polynucleotides can, if desired, include an RNA cleaving group, a cholesteryl group, a reporter group, an intercalator, a group for improving the pharmacokinetic properties of the polynucleotide, and/or a group for improving the pharmacodynamic properties of the polynucleotide.

Those of skill in the art understand that polynucleotides complementary to the coding strand of polynucleotides of the invention can be employed to inhibit expression of the polypeptides of the invention, which may be of interest for research or therapeutic purposes. Accordingly, the nucleic acids of the invention include such “antisense polynucleotides,” and the phrase “polynucleotide encoding a polypeptide of the invention” is intended to include such antisense molecules.

B. Vectors

A polynucleotide of the invention can be incorporated into a vector for propagation and/or expression in a host cell. Such vectors typically contain a replication sequence capable of effecting replication of the vector in a suitable host cell (i.e., an origin of replication) as well as sequences encoding a selectable marker, such as an antibiotic resistance gene. Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art.

If the vector is intended for expression of a polypeptide, the vector includes one or more control sequences capable of effecting and/or enhancing the expression of an operably linked polypeptide coding sequence. Control sequences that are suitable for expression in prokaryotes, for example, include a promoter sequence, an operator sequence, and a ribosome binding site. Control sequences for expression in eukaryotic cells include a promoter, an enhancer, and a transcription termination sequence (i.e., a polyadenylation signal).

An expression vector according to the invention can also include other sequences, such as, for example, nucleic acid sequences encoding a signal sequence or an amplifiable gene. A signal sequence can direct the secretion of a polypeptide fused thereto from a cell expressing the protein. In the expression vector, nucleic acid encoding a signal sequence is linked to a polypeptide coding sequence so as to preserve the reading frame of the polypeptide coding sequence. The inclusion in a vector of a gene complementing an auxotrophic deficiency in the chosen host cell allows for the selection of host cells transformed with the vector.

A vector of the present invention is produced by linking desired elements by ligation at convenient restriction sites. If such sites do not exist, suitable sites can be introduced by standard mutagenesis (e.g., site-directed or cassette mutagenesis) or synthetic oligonucleotide adaptors or linkers can be used in accordance with conventional practice.

Viral vectors are of particular interest for use in delivering polynucleotides of the invention to a cell or organism, followed by expression of the encoded protein. Widely used vector systems include, but are not limited to adenovirus, adeno associated virus, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088. Adenoviral vectors suitable for use in the invention are also commercially available. For example, the Adeno-X™ Tet-Off™ gene expression system, sold by Clontech, provides an efficient means of introducing inducible heterologous genes into most mammalian cells.

Adeno-associated virus (AAV)-based vectors used to transduce cells with polynucleotides, e.g., in the in vitro production of polynucleotides and peptides, and in vivo and ex vivo gene therapy procedures are described, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include those derived from herpes virus, lentivirus, and vaccinia virus.

C. Host Cells

The present invention also provides a host cell containing a vector of this invention. A wide variety of host cells are available for propagation and/or expression of vectors. Examples include prokaryotic cells (such as E. coli and strains of Bacillus, Pseudomonas, and other bacteria), yeast or other fungal cells (including S. cerevesiae and P. pastoris), insect cells, plant cells, and phage, as well as higher eukaryotic cells (such as human embryonic kidney cells and other mammalian cells). Host cells according to the invention include cells in culture and cells present in live organisms, such as transgenic plants or animals or cells into which a gene therapy vector has been introduced.

A vector of the present invention is introduced into a host cell by any convenient method, which will vary depending on the vector-host system employed. Generally, a vector is introduced into a host cell by transformation (also known as “transfection”) or infection with a virus (e.g., phage) bearing the vector. If the host cell is a prokaryotic cell (or other cell having a cell wall), convenient transformation methods include the calcium treatment method described by Cohen, et al. (1972) Proc. Natl. Acad. Sci., USA, 69:2110-14. If a prokaryotic cell is used as the host and the vector is a phagemid vector, the vector can be introduced into the host cell by infection. Yeast cells can be transformed using polyethylene glycol, for example, as taught by Hinnen (1978) Proc. Natl. Acad. Sci, USA, 75:1929-33. Mammalian cells are conveniently transformed using the calcium phosphate precipitation method described by Graham, et al. (1978) Virology, 52:546 and by Gorman, et al. (1990) DNA and Prot. Eng. Tech., 2:3-10. However, other known methods for introducing DNA into host cells, such as nuclear injection, electroporation, and protoplast fusion also are acceptable for use in the invention.

Recombinant Production Methods

Host cells transformed with expression vectors can be used to express the polypeptides encoded by the polynucleotides of the invention. Expression entails culturing the host cells under conditions suitable for cell growth and expression and recovering the expressed polypeptides from a cell lysate or, if the polypeptides are secreted, from the culture medium. In particular, the culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number; factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in expressing the polypeptides of the invention. These include, but are not limited to, the use of a shaken flask, a fluidized bed bioreactor, a roller bottle culture system, and a stirred tank bioreactor system. Cell culture can be carried out in a batch, fed-batch, or continuous mode.

Methods for recovery of recombinant proteins produced as described above are well-known and vary depending on the expression system employed. A polypeptide including a signal sequence can be recovered from the culture medium or the periplasm. Polypeptides can also be expressed intracellularly and recovered from cell lysates.

The expressed polypeptides can be purified from culture medium or a cell lysate by any method capable of separating the polypeptide from one or more components of the host cell or culture medium. Typically, the polypeptide is separated from host cell and/or culture medium components that would interfere with the intended use of the polypeptide. As a first step, the culture medium or cell lysate is usually centrifuged or filtered to remove cellular debris. The supernatant is then typically concentrated or diluted to a desired volume or diafiltered into a suitable buffer to condition the preparation for further purification.

The polypeptide can then be further purified using well-known techniques. The technique chosen will vary depending on the properties of the expressed polypeptide. If, for example, the polypeptide is expressed as a fusion protein containing an epitope tag or other affinity domain, purification typically includes the use of an affinity column containing the cognate binding partner. For instance, polypeptides fused with green fluorescent protein, hemagglutinin, or FLAG epitope tags or with hexahistidine or similar metal affinity tags can be purified by fractionation on an affinity column.

Compositions

The anti-H2B-r36p antibodies, as well as the transcription factor and H2B polypeptides of the invention or polynucleotides encoding them, can formulated for use in assays and/or administration to cells, tissues, or organisms. The compositions optionally contain other components, including, for example, a storage solution, such as a suitable buffer, e.g., a physiological buffer. In a preferred embodiment, the composition is a pharrnaceutical composition and the other component is a pharmaceutically acceptable carrier, such as are described in Remington's Pharmaceutical Sciences (1980) 16th editions, Osol, ed., 1980.

Screening for Modulators of Phosphorylation of Histone H2B at Residue 36

The role of transcription factor phosphorylation of histone H2B (H2B) at residue 36 in regulating genes that influence cell proliferation makes the transcription factor-H2B interaction an attractive target for use in screening for agents that modulate cell proliferation. Similarly, the role of the physical association of histone H2B that is phosphorylated at residue 36 (H2B-r36p) with the promoters of genes that influence the cell cycle in the transcriptional activation of such genes can also be exploited as a target in screening for cell proliferation modulators. Accordingly, the invention provides prescreening and screening methods aimed at identifying agents that either inhibit or stimulate cell proliferation. The prescreening/screening methods of the invention are generally, although not necessarily, carried out in vitro. Accordingly, screening assays are generally carried out, for example, using purified or partially purified components, in cell lysates, in cultured cells, or in other biological samples.

A. Prescreening Methods

1. Prescreening Based on Binding to H2B Polypeptides or Polynucleotides

The prescreening methods are based on screening test agents for specific binding, either to an H2B polypeptide or polynucleotide or to a transcription factor polypeptide or polynucleotide. Agents that specifically bind to H2B polypeptides have the potential to influence transcription factor-mediated phosphorylation of H2B at residue 36 and/or promoter association of H2B-r36p. Accordingly, agents that specifically bind to H2B polypeptides are candidate modulators of cell proliferation.

In one embodiment, therefore, a prescreening method of the invention entails contacting a test agent with a H2B polypeptide comprising H2B or a fragment thereof. Specific binding of the test agent to the H2B polypeptide is then detected. Suitable H2B polypeptides include H2B residue 36, the H2B residues being numbered according to human H2B. H2B residue 36 can be a serine or a threonine. The H2B polypeptides can be from any organism, as described above for H2B derivatives of the invention, and these derivatives are suitable for use in this prescreening method.

Agents that specifically bind to H2B polynucleotides have the potential to decrease or increase the expression of H2B polypeptides, which can potentially inhibit or stimulate, respectively, transcription factor-mediated phosphorylation of H2B at residue 36 and/or promoter association of H2B-r36p. Thus, in an alternative embodiment, the test agent can be contacted with a polynucleotide encoding the H2B polypeptide to screen for agents that affect H2B expression, followed by detection of specific binding of the test agent to the H2B polynucleotide.

Such prescreening is generally most conveniently accomplished with a simple in vitro binding assay. Means of assaying for specific binding of a test agent to a polypeptide or polynucleotide are well known to those of skill in the art. In preferred binding assays, the H2B polypeptide of polynucleotide is immobilized and exposed to a test agent (which can be labeled), or alternatively, the test agent(s) are immobilized and exposed to the H2B polypeptide or polynucleotide (which can be labeled). The immobilized moiety is then washed to remove any unbound material and the bound test agent or bound H2B polypeptide or polynucleotide is then detected. To prescreen large numbers of test agents, high throughput assays are generally preferred.

2. Prescreening Based on Binding to Transcription Factor Polypeptides or Polynucleotides

Agents that specifically bind to polypeptides comprising transcription factors capable of phosphorylating H2B at residue 36 have the potential to influence transcription factor-mediated phosphorylation of H2B at residue 36. Accordingly, agents that specifically bind to transcription factor polypeptides are candidate modulators of cell proliferation.

In one embodiment, therefore, a prescreening method of the invention entails contacting a test agent with a transcription factor polypeptide comprising a transcription factor or a fragment thereof. Specific binding of the test agent to the transcription factor polypeptide is then detected. Suitable transcription factor polypeptides include a double bromodomain kinase and can phosphorylate H2B at H2B residue 36. The transcription factor polypeptides can be from any organism, as described above for transcription factor derivatives of the invention, and these derivatives are suitable for use in this prescreening method.

Agents that specifically bind to transcription factor polynucleotides have the potential to decrease or increase the expression of transcription factor polypeptides, which can potentially inhibit or stimulate, respectively, transcription factor-mediated phosphorylation of H2B at residue 36. Thus, in an alternative embodiment, the test agent can be contacted with a polynucleotide encoding the transcription factor polypeptide to screen for agents that affect transcription factor expression, followed by detection of specific binding of the test agent to the transcription factor polynucleotide.

Such prescreening is generally most conveniently accomplished with a simple in vitro binding assay. Means of assaying for specific binding of a test agent to a polypeptide or polynucleotide are well known to those of skill in the art and are briefly described above with respect to prescreening based on binding to H2B polypeptides.

B. Screening Methods

Another aspect of the invention is a method of screening for a modulator of phosphorylation of histone H2B (H2B) at residue 36. The method entails contacting a test agent with a medium including H2B, or a fragment thereof, and a transcription factor, or a fragment thereof, under conditions suitable for phosphorylation of H2B residue 36, followed by detection of phosphorylation of H2B residue 36. The H2B, or fragment thereof, includes H2B residue 36, the H2B residues being numbered according to human H2B. H2B residue 36 can be a serine or a threonine.

The transcription factor, or fragment thereof, includes a double bromodomain kinase and phosphorylates H2B at H2B residue 36.

Both polypeptides can be from any organism, as described above for H2B and transcription factor derivatives of the invention, and these derivatives are suitable for use in this screening method. The polypeptides selected for a particular assay can be from the same or different organisms, provided the transcription factor employed is capable of phosphorylating the H2B employed. Generally, mammalian polypeptides are preferred, and human polypeptides are more preferred for use in the screening method.

The medium can be any physiological medium in which the transcription factor kinase is active, and the assay is carried out under conditions suitable for phosphorylation of H2B residue 36. Cell-free systems or intact cells can be employed. In the latter case, assays may be carried out in vitro (e.g., in cultured cells) or in vivo. Both are illustrated in the Examples herein. In preferred embodiments, assays are carried out using intact chromatin, i.e., to determine the effect of the test agent on the phosphorylation of H2B in its chromatin-associated state.

After allowing phosphorylation, if any, to occur phorphorylation of H2B residue 36 is detected using any convenient method, such as any of those described above or illustrated in the Examples. Immunoassays are generally preferred.

Any phosphorylation of H2B residue 36 observed in the reaction including the test agent (“test reaction”) is preferably compared with phosphorylation of H2B residue 36 in a control reaction, i.e., one carried out in the absence of test agent or in the presence of a lower amount of test agent. A reduction in phosphorylation in the test reaction, as compared to the control reaction, indicates that the test agent is a candidate inhibitor of cell proliferation; whereas an increase in phosphorylation in the test reaction, as compared to the control reaction, indicates that the test agent is a candidate stimulator of cell proliferation. Inhibitors of cell proliferation are useful in treating diseases characterized by inappropriate or undesirable cell proliferation, such as cancer. Agents that stimulate cell proliferation are useful for applications where cell proliferation is desired, such as wound healing, regeneration of bone or other tissues, etc.

An alterative screening method of the invention is based on screening for a modulator of promoter association of histone H2B (H2B) that is phosphorylated at residue 36 (H2B-r36p). The method entails contacting a test agent with cells including H2B, or a fragment thereof, and a transcription factor, or a fragment thereof, and then determining whether H2B-r36p is physically associated with the promoter of a gene. The H2B, or fragment thereof, includes H2B residue 36, the H2B residues being numbered according to human H2B. H2B residue 36 can be a serine or a threonine.

The transcription factor, or fragment thereof, includes a double bromodomain kinase and phosphorylates H2B at H2B residue 36.

The cells can be from any organism. Generally, however, mammalian cells are preferred, and human cells are more preferred for use in the screening method. Assays may be carried out in vitro (e.g., in cultured cells) or in vivo. Both are illustrated in the Examples herein.

Promoter association of H2B-r36p with the gene of interest is determined using any convenient method, such as any of those described above or illustrated in the Examples. In vivo cross-linked chromatin immunoprecipitation is generally preferred. Promoter association with any gene may be assayed. In preferred embodiments, assays may be performed to determine association with the promoters of, e.g., cell cycle genes, tissue differentiation genes, or oncogenes.

Any promoter association of H2B-r36p observed in the reaction including the test agent (“test reaction”) is preferably compared with the promoter association of H2B-r36p in a control reaction, i.e., one carried out in the absence of test agent or in the presence of a lower amount of test agent. A reduction in promoter association in the test reaction, as compared to the control reaction, indicates that the test agent is a candidate inhibitor of cell proliferation; whereas an increase in promoter association in the test reaction, as compared to the control reaction, indicates that the test agent is a candidate stimulator of cell proliferation.

C. Test Agent Databases

In a preferred embodiment, generally involving the screening of a large number of test agents, the screening method includes the recordation of any test agent that induces a difference in H2B phosphorylation or promoter association of H2B-r33p in a database of candidate modulator of cell proliferation. Separate databases can be employed for candidate inhibitors and candidate stimulators of proliferation. In particular embodiments, it may be desirable to screen for one effect and record only the test agents that produce that effect in a database.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

D. Further Study of Test Agents

In preferred embodiments, the methods of the invention include further study of one or more test agents to determine whether the test agent inhibits or stimulates cell proliferation. The degree of cell proliferation observed in the presence of a test agent is preferably compared with the degree of cell proliferation observed in the absence of the test agent or in the presence of a lower amount of test agent. Cell proliferation assays are well known, and any standard proliferation assay can be employed in the invention. Such assays can be carried out in vivo or in vitro, although in vitro assays are generally preferred. In a commercially available assay, cells are quantified by an MTS (3-(4,5-dimethylthiazol-2-yl)-5-(3-carboxylmethoxyphenyl)-2-(4-sulfophenyl )-2H-tetrazolium, inner salt) conversion assay, where MTS conversion to a formazan is proportional to cell number and can be followed by absorbance at 490 nM (Cell Titer 96 AQueous One Solution Cell Proliferation Assay, Promega, Madison, Wis., USA).

Modulation of Cell Proliferation

1. Inhibition of Cell Proliferation

Inhibition of phosphorylation of histone H2B (H2B) at residue 36 (wherein H2B residues are numbered according to human H2B) leads to G2/M phase arrest. This phenomenon can be exploited in any setting in which cells are proliferating inappropriately or where it is otherwise desirable to inhibit cell proliferation. In addition, the effects of inhibiting H2B-r36 phosphorylation are of significant research interest. Accordingly, the invention provides a method of inhibiting cell proliferation based on reducing or preventing phosphorylation of H2B at residue 36. The method entails contacting cells containing a transcription factor and histone H2B with an effective amount of an inhibitor.

The transcription factor is one that includes a double bromodomain kinase and phosphorylates H2B at H2B residue 36. Suitable transcription factors of this type include TAF1, which is exemplified herein with the human and Drosophila TAF1s.

The inhibitor is any inhibitor that reduces or prevents phosphorylation of H2B-r36, such that the phosphorylation observed in the presence of the inhibitor is less than that observed in the absence of inhibitor (or in the presence of a lower amount of inhibitor). Suitable inhibitors can inhibit phosphorylation, for example, by binding to the kinase domain of the transcription factor or to the H2B domain containing residue 36; binding near either of these domains and sterically hindering access to the domain; or binding to a different region of either protein and inducing a conformational change in the relevant domain. In various embodiments, the inhibitor reduces phosphorylation of H2B-r36 by at least about 10, 20, 30, 40, 50, 60, 70, 80, 90, and 95 percent, as determined by a kinase assay, such as that described in Example 1.

The inhibitor is preferably suitable for administration to cells and/or organisms, e.g., not unduly toxic. In addition, the inhibitor is preferably a molecule that can be delivered across the cell membrane to interact with TAF1 and/or histone or the genes encoding these proteins. Inhibitors suitable for use in the invention can be identified using one or more of the novel screening methods of the invention.

Any cells that express a suitable transcription factor and histone H2B can be employed in the method. The cell is typically, although not necessarily, one that expresses these two proteins endogenously. The method generally employs animal cells, typically cells from eukaryotes; particularly vertebrates; more particularly mammals, such as dogs, cats, sheep, cattle and pigs; and most particularly primates, such as humans, chimpanzees, gorillas, macaques, and baboons, and rodents such as mice, rats, and guinea pigs.

The cells are generally contacted with inhibitor under physiological conditions. The duration of contact with the inhibitor can vary, depending on the particular application of the method. The duration of contact can range from minutes to days or longer. For research applications, the inhibitor is typically contacted with cells for, e.g., about 30 mins.; or about 1, about 3, about 6, or about 12 hours; or about 1, about 2, about 5, about 10, or about 15 days.

Contact of the inhibitor with cells can be achieved directly, i.e., by administering a composition containing the inhibitor to the cells, or indirectly, e.g., by administering a composition containing a polynucleotide encoding an inhibitor polypeptide to the cells. In the latter embodiment, this administration results in the introduction of the polynucleotide into one or more cells and the subsequent expression of the polypeptide in an amount sufficient to reduce H2B-r36 phorphorylation.

This method can be carried out in vitro, i.e., in cells or tissues that are not part of an organism, or in vivo, in cells that are part of an organism. In one embodiment, cells are contacted in vitro in with an effective amount of an inhibitor (or a polynucleotide encoding the inhibitor).

Alternatively, cells can be contacted in vivo with a inhibitor by administering a composition containing the inhibitor (or a polynucleotide encoding the inhibitor) directly to a subject having, or at risk for, a condition that can be ameliorated by inhibiting cell proliferation, for example, a cancer patient. Suitable compositions can be formulated as described above.

2. Stimulation of Cell Proliferation

Phosphorylation of histone H2B (H2B) at residue 36 (wherein H2B residues are numbered according to human H2B) contributes to cell proliferation. This phenomenon can be exploited in any setting in which it is desirable to stimulate cell proliferation. In addition, the effects of stimulating H2B-r36 phosphorylation are of significant research interest. Accordingly, the invention provides a method of stimulating cell proliferation based on increasing phosphorylation of H2B at residue 36. The method entails contacting cells containing a transcription factor and histone H2B with an effective amount of a stimulatory agent (i.e., one that increases H2B-r36 phosphorylation).

The transcription factor is one that includes a double bromodomain kinase and phosphorylates H2B at H2B residue 36. Suitable transcription factors of this type include TAF1, which is exemplified herein with the human and Drosophila TAF1s.

The stimulatory agent is any agent that increases phosphorylation of H2B-r36, such that the phosphorylation observed in the presence of the stimulatory agent is greater than that observed in its absence (or in the presence of a lower amount of stimulatory agent). In various embodiments, the stimulatory agent increases phosphorylation of H2B-r36 by at least about 10, 20, 30, 40, 50, 60, 70, 80, 90, and 95 percent, as determined by a kinase assay, such as that described in Example 1.

The stimulatory agent is preferably suitable for administration to cells and/or organisms, e.g., not unduly toxic. In addition, the agent is preferably a molecule that can be delivered across the cell membrane to interact with TAF1 and/or histone or the genes encoding these proteins. Stimulatory agents suitable for use in the invention can be identified using one or more of the novel screening methods of the invention.

The considerations affecting choice of cell and how contact with the stimulatory agent is carried out are the same as described above for inhibitors of H2B-r36 phosphorylation.

This method can be performed in vitro or in vivo. In one embodiment, cells are contacted in vitro in with an effective amount of a stimulatory agent (or a polynucleotide encoding the stimulatory agent).

Alternatively, cells can be contacted in vivo with a stimulatory agent by administering a composition containing the stimulatory agent (or a polynucleotide encoding the stimulatory agent) directly to a subject having a condition treatable by induction of cell proliferation, for example, a subject with a non-healing or poorly healing wound or in need of bone regeneration or regeneration of other tissues Suitable compositions can be formulated as described above.

3. Administration

For in vitro applications, cells are contacted with an inhibitor of the invention simply by adding the inhibitor or the polynucleotide encoding the inhibitor directly to the medium of cultured cells or directly to tissues.

Methods for in vivo administration do not differ from known methods for administering drugs or therapeutic polypeptides, peptides, or polynucleotides encoding them. Suitable routes of administration include, for example, topical, intravenous, intraperitoneal, intracerebral, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

4. Dose

The dose of inhibitor is sufficient to inhibit cell proliferation without undue toxicity. For in vivo applications, the dose of inhibitor depends, for example, upon the therapeutic objectives, the route of administration, and the condition of the subject. It is routine for the clinician to titer the dosage and modify the route of administration as required to obtain the optimal therapeutic effect. Generally, the clinician begins with a low dose and increases the dosage until the desired therapeutic effect is achieved. Starting doses for a given inhibitor can be extrapolated from in vitro data.

### EXAMPLES

The following examples are offered to illustrate, but not to limit, the claimed invention.

### Example 1

### TAF1 Activates Transcription by Phophorylation of Serine 33 in Drosophila melanogaster Histone H2B

Summary

Dynamic changes in chromatin structure, induced by post-translational modification of histones, play a fundamental role in regulating eukaryotic transcription. This example establishes that histone H2B is phosphorylated at evolutionarily conserved serine 33 (H2B-S33) by the COOH-terminal kinase domain (CTK) of the Drosophila TFIID subunit TAF1. Phosphorylation of H2B-S33 at the promoter of the cell cycle regulatory gene string and the segmentation gene giant coincides with transcriptional activation. Elimination of TAF1 CTK activity in Drosophila cells and embryos reduces transcriptional activation and phosphorylation of H2B-S33. These data reveal that H2B-S33 is a physiological substrate for the TAF1 CTK and that H2B-S33 phosphorylation is essential for transcriptional activation events that promote cell cycle progression and development.

Results

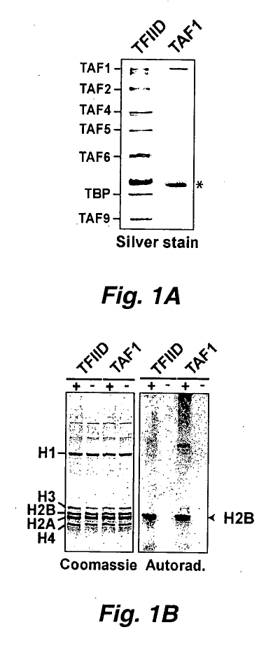

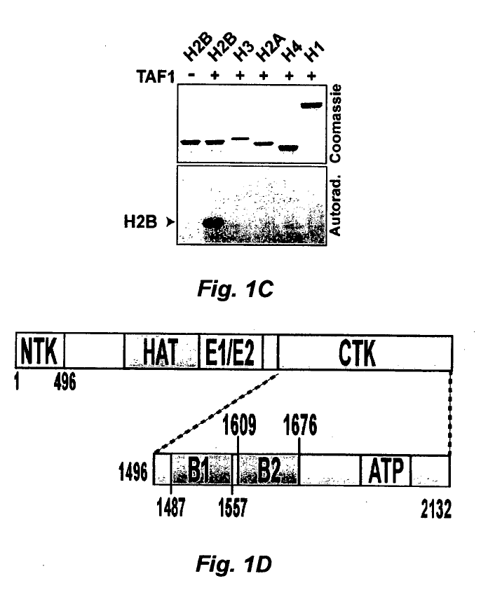

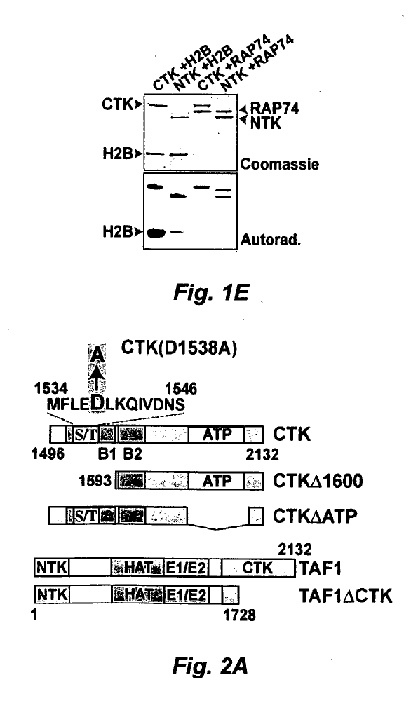

Using an in vitro kinase assay, it was found that the Drosophila general transcription factor (GTF) TFIID phosphorylates histone H2B but not H1, H2A, H3 or H4 (FIGS. 1A and B) (8). TFIID is a multi-protein complex composed of the TATA-box binding protein (TBP) and numerous TBP associated factors (TAFs) (9). TFIID functions during transcription initiation by nucleating assembly of GTFs and RNA polymerase II at the promoter. TAF1 (formerly TAF_{II}250) is the only TFIID subunit that possesses kinase activity, suggesting that it phosphorylates H2B (10-12). In fact, recombinant TAF1 and denatured/renatured recombinant TAF1 phosphorylated H2B in vitro, demonstrating that TAF1 has intrinsic, H2B-specific kinase activity (FIGS. 1B and C) (8). Collectively, these results indicate that TAF1 alone and TAF1 in the context of TFIID phosphorylate H2B.

TAF1 contains two kinase domains, an NH₂-terminal (NTK; amino acids 1-496) and a COOH-terminal (CTK; amino acids 1496-2132) domain (10) (FIG. 1D). In vitro, the NTK and CTK autophosphorylate and the NTK trans-phosphorylates the RAP74 subunit of the GTF TFIIF. To determine which domain phosphorylates H2B, the NTK and CTK were assayed separately in vitro. In contrast to the NTK, the CTK did not phosphorylate RAP74 but strongly phosphorylated H2B, indicating that the CTK possesses H2B-specific kinase activity (FIG. 1E).

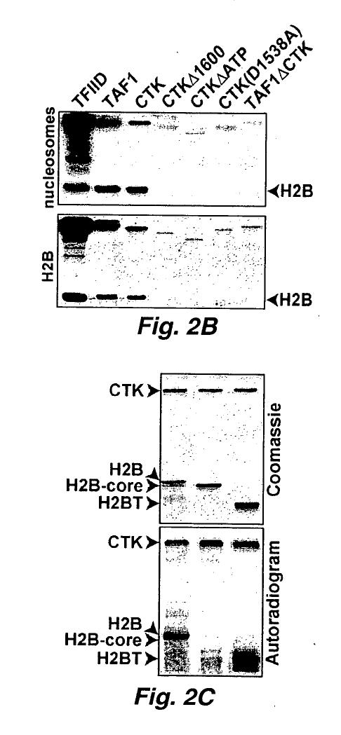

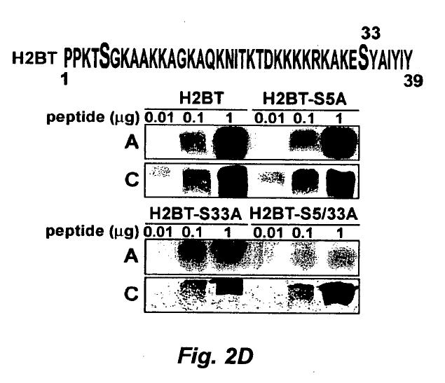

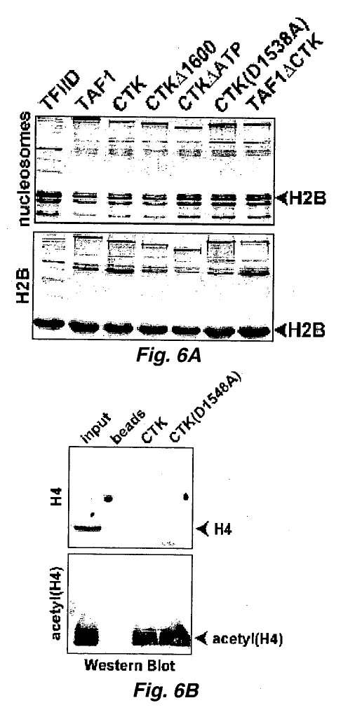

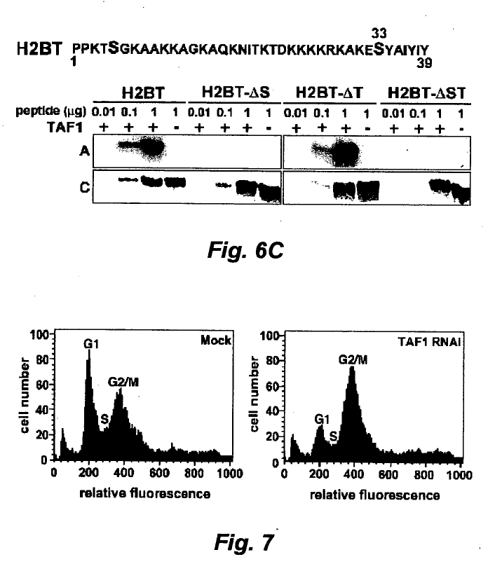

Protein kinases contain two essential functional motifs, an ATP-binding motif and an amino acid-specific kinase motif. Computational sequence comparison analyses identified a putative serine/threonine (S/T) kinase motif (amino acids 1534-1546) and two tandem ATP-binding domains (amino acids 1747-1780) in the CTK (FIGS. 2A and 5) (8, 13). To test whether the identified motifs mediate H2B phosphorylation, in vitro kinase assays were performed using CTK polypeptides lacking the S/T-kinase motif (CTKΔ1600) or the ATP binding motifs (CTKΔATP). Relative to the wild type CTK, CTKΔ1600 and CTKΔATP weakly phosphorylated H2B (FIGS. 2B and 6A). To confirm the role of the S/T-kinase motif, a catalytically important aspartic acid was mutated to an alanine (D1538A) in the motif (FIGS. 2A and 5) (13). Like CTKΔ1600, CTK(D1538A) exhibited weak autophosphorylation and H2B trans-phosporylation activities (FIG. 2B). Interestingly, the S/T-kinase motif is located in the first bromodomain of the double bromodomain module (DBD), which binds acetylated lysines (FIG. 2A) (14). However, CTK(D1538A) retains the ability to bind acetylated H4 in vitro indicating that the introduced mutation disrupts kinase activity rather than acetylated lysine-based substrate recognition (FIG. 6B). Thus, the identified S/T-kinase and ATP-binding motifs of the TAF1 CTK are essential for H2B phosphorylation.

To identify H2B residue(s) phosphorylated by the CTK, the CTK was tested to see if it phosphorylates the NH₂-terminal tail of Drosophila H2B (amino acids 1-39; H2BT) or the tailless H2B core domain (amino acids 40-123). It was found that the CTK phosphorylated H2BT but not the H2B core domain (FIG. 2C). Next, to pinpoint which residue(s) in H2BT is phosphorylated, mutant H2BT peptides were generated in which alanines replaced all or individual serines or threonines. The CTK did not phosphorylate peptides lacking all serines, suggesting that it phosphorylates either serine 5 (H2B-S5) or 33 (H2B-S33) (FIG. 6C). To test this, H2BT peptides were used as substrates that contained alanines in place of H2B-S5, H2B-S33, or both (H2BT-S5A, H2BT-S33A, and H2BT-S5/33A, respectively). The CTK phosphorylated H2BT-S5A but not H2BT-S33A or H2BT-S5/33A indicating that H2B-S33 is the target of the CTK (FIG. 2D).

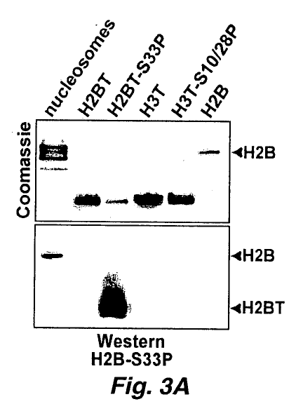

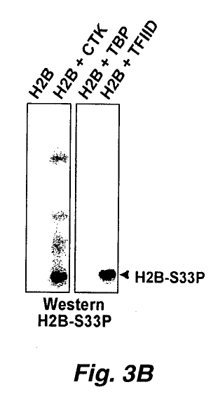

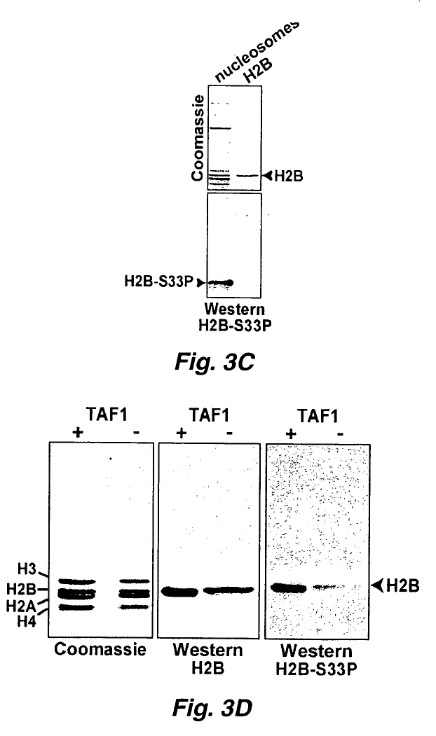

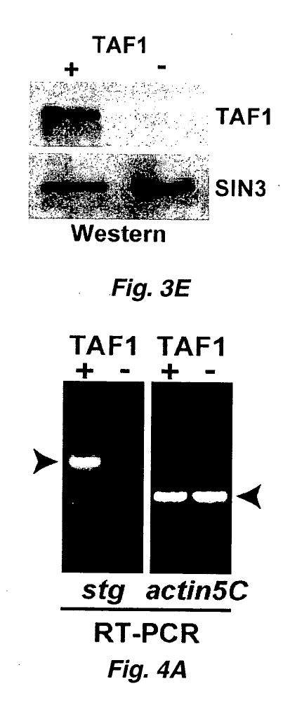

To investigate whether H2B-S33 is phosphorylated in vivo, a polyclonal antibody recognizing phosphorylated H2B-S33 (H2B-S33P) was raised (8). On Western blots, the antibody recognized H2BT containing H2B-S33P but not recombinant, unphosphorylated H2B or an H3 peptide (amino acids 1-32) containing phosphorylated serine 10 and 28 (FIG. 3A). In addition, the H2B-S33P antibody recognized H2BT and recombinant H2B that was phosphorylated in vitro by the CTK or TFIID, indicating that the antibody specifically recognizes phosphorylated H2B-S33 (FIG. 3B). The H2B-S33 antibody also recognized a protein with molecular weight similar to H2B from histone preparations from Drosophila embryos or S2 cells, providing evidence that H2B-S33 is a target for phosphorylation in vivo (FIGS. 3C and D) (15, 16). To determine whether TAF1 mediates H2B-S33 phosphorylation in vivo, RNA interference (RNAi) was used to eliminate TAF1 expression in S2 cells (8). By Western blot analysis, both TAF1 expression and H2B-S33 phosphorylation were reduced in TAF1 RNAi cells, compared to mock RNAi cells, suggesting that TAF1 is a major H2B-S33 kinase in vivo (FIGS. 3D and E).

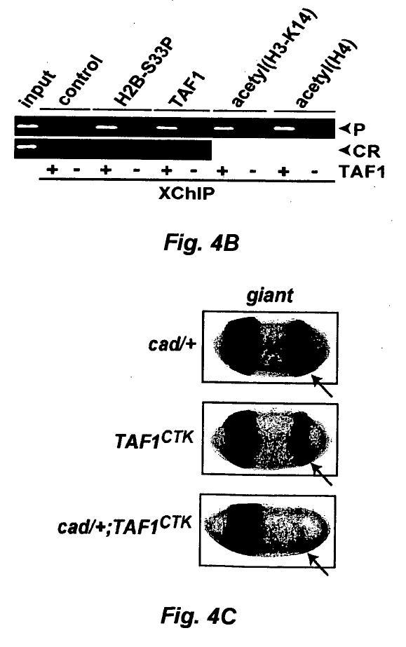

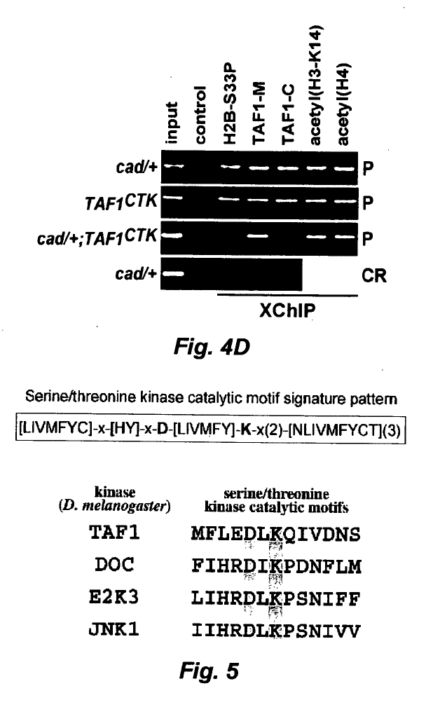

Flow cytometry analysis of TAF1 RNAi cells revealed that loss of TAF1 results in G2/M phase cell cycle arrest (FIG. 7). To test the hypothesis that TAF1 controls the transcription of genes whose activities contribute to G2/M progression, microarray expression profiling and RT-PCR were used to monitor transcription in mock and TAF1 RNAi cells. Both methods showed that transcription of string (stg), which encodes a Drosophila homolog of yeast Cdc25, was reduced (FIG. 4A) (8). The Stg protein phosphatase is predominantly expressed during G2 and activates the cell cycle by dephosphorylating Cdc2 (17). That the loss of stg from S2 cells by RNAi causes G2/M arrest, indicates that TAF1 regulates G2/M progression by activating stg transcription (18).

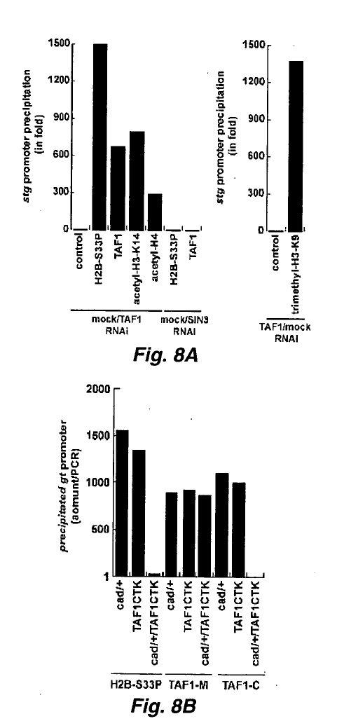

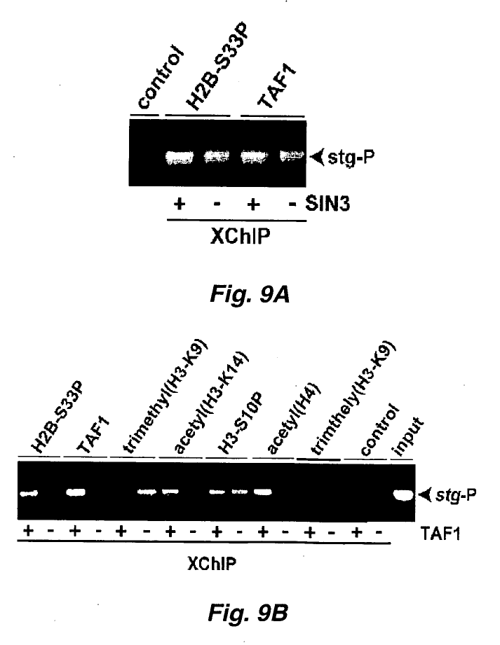

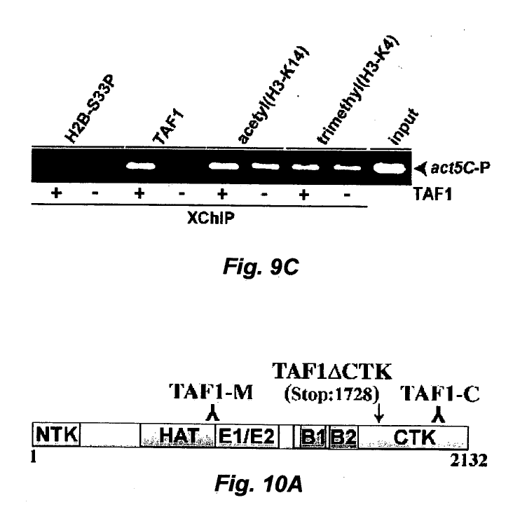

Chromatin immunoprecipitation (XChIP) was used to establish whether there is a direct correlation between transcriptional activation of stg and TAF1-mediated phosphorylation of H2B-S33 at the stg promoter (15). Cross-linked chromatin was isolated from mock and TAF1 RNAi S2 cells and immunoprecipitated with TAF1 or H2B-S33P antibodies. Immunoprecipitated DNA was purified and used as a template for PCR to detect the stg promoter or coding region and actin5C promoter. TAF1 and H2B-S33P antibodies precipitated the stg promoter, but not the coding region, from mock cells, and precipitated neither the stg promoter, nor the coding region from TAF1 RNAi cells (FIGS. 4B and 8). In contrast, TAF1 is not essential for actin5C transcription and H2B-S33P antibodies do not precipitate the actin5C promoter (FIGS. 4A and 9A). Thus, the transcriptional dependence of a gene for TAF1 is correlated with H2B-S33 phosphorylation not with TAF1 association.

To distinguish whether loss of H2B-S33 phosphorylation at the stg promoter is due directly to loss of TAF1 or indirectly to G2/M arrest, XChIP analysis was performed on S2 cells arrested in G2/M by RNAi of the SIN3 transcriptional corepressor. Stg transcription is repressed in SIN3 RNAi cells, yet the stg promoter remains associated with H2B-S33P and TAF1, indicating that loss of H2B-S33 phosphorylation in TAF1 RNAi cells is due to elimination of TAF1 rather than G2/M arrest (FIG. 9B) (18).

In addition to kinase domains, TAF1 has a histone acetyltransferase (HAT) domain that acetylates H3 lysine 14 (H3-K14) and unidentified lysines in H4 in vitro (19). XChIP analysis detected acetylated H3-K14 and H4 at the transcriptionally active stg promoter in mock RNAi cells but not at the inactive stg promoter in TAF1 RNAi cells (FIGS. 4B and 9C). In contrast, TAF1-independent histone modifications did not correlate with activation of stg in mock and TAF1 RNAi cells (FIG. 9C). Taken together, these results indicate that TAF1-mediated phosphorylation of H2B-S33 and acetylation of H3 and H4 potentiate transcriptional activation in Drosophila cells.

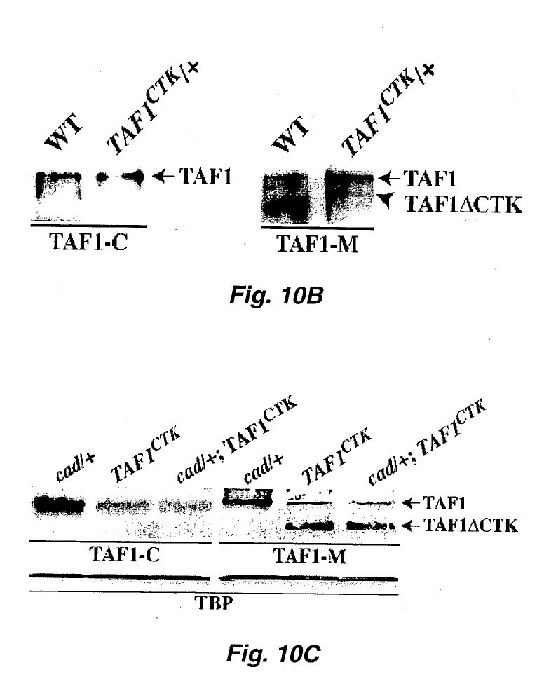

A recessive lethal TAF1 allele, TAF1^{CTK}, which contains a nonsense mutation at amino acid 1728, deleting the CTK downstream of the DBD, was used to investigate the role of TAF1-mediated phosphorylation of H2B-S33 during fly development (FIGS. 2A and 10A) (8). The corresponding protein (TAF1ΔCTK) is expressed in Drosophila but presumably does not have CTK activity, as it does not phosphorylate H2B in vitro (FIGS. 2B and 10B and C). In situ hybridization was used to monitor transcription in embryos homozygous mutant for TAF1^{CTK} and heterozygous mutant for the maternal activator Caudal (Cad) (20). In this genetic background, transcription of the gap gene giant (gt) was reduced (FIG. 4C). Gt is transcribed in two domains along the anterior-posterior axis of blastoderm stage embryos. Transcription of the posterior gt domain (pgt) is Cad-dependent while transcription of the anterior gt domain (agt) is Cad-independent. Relative to controls (cad/+ or TAF1^{CTK}), pgt transcription was reduced in cad/+; TAF1^{CTK} embryos (FIG. 4C).

XChIP analysis was used to examine whether TAF1-mediated phosphorylation of H2B-S33 contributes to pgt transcription. Cross-linked chromatin was isolated from the posterior halves of cad/+; TAF1^{CTK} and control embryos, and immunoprecipitated using antibodies to H2B-S33P, acetylated histones, or TAF1. PCR analysis detected H2B-S33P at the transcriptionally active gt promoter in control embryos but not at the transcriptionally repressed promoter in cad/+;TAF1^{CTK} embryos (FIGS. 4D and 8). To monitor TAF1 binding, two antibodies were used, TAF1-M and TAF1-C, which recognize the middle domain and the CTK of TAF1, respectively (FIG. 10A). Both antibodies precipitated the gt promoter from control embryos, indicating that TAF1ΔCTK and maternally-contributed, wild type TAF1 are present at the gt promoter in the pgt (FIGS. 4D and 8). In contrast, while the TAF1-M antibody precipitated the gt promoter from cad/+; TAF1^{CTK} embryos, TAF1-C did not. Since TAF1ΔCTK is present at a higher concentration in cad/+; TAF1^{CTK} embryos than maternal TAF1, this result indicates that TAF1ΔCTK is preferentially recruited to the gt promoter in the pgt (FIGS. 4D and 10C). This result is supported by the presence of TAF1-mediated histone acetylation at the transcriptionally silent gt promoter. Thus, TAF1-mediated phosphorylation of H2B-S33 contributes to transcriptional activation during Drosophila embryogenesis.

Serine 33 is the only evolutionarily conserved serine or threonine in the NH₂-terminus of metazoan H2Bs (FIG. 11). In the crystal structure of the Xenopus laevis nucleosome, the equivalent serine links the H2B DNA-binding NH₂-terminal tail to the histone fold domain (3, 21). Thus, replacing the hydroxyl group on serine 33 with a bulkier, negatively charged phosphate group may drastically affect H2B tail interactions with DNA. This is important because the H2B tail regulates nucleosome mobility. Deletion of the tail bypasses the requirement for the SWI/SNF nucleosome-remodeling complex in yeast, and the tail is critical for maintaining the position of histone octamers in in vitro sliding assays (22, 23). These findings support a model in which TAF1-mediated phosphorylation of H2B-S33 disrupts DNA-histone interactions resulting in local decondensation of chromatin. Decondensation may trigger chromatin remodeling and formation of a chromatin structure that facilitates assembly of other GTFs at a promoter, a function that is primarily attributed to TFIID (1, 2, 10).

The data indicate that the S/T-kinase motif of the CTK is located in the DBD. In the crystal structure of the DBD, the position of the S/T-kinase motif does not overlap with the acetylated lysine-binding surface of the DBD suggesting that it is an independent functional unit of the DBD (14). Members of the fsh/RING3 (BET) family of DBD proteins have kinase activity, suggesting that TAF1 is a member of a kinase family whose catalytic motif resides within the DBD (24, 25).

Phosphorylation of H2B-S33 by TAF1 is essential for transcriptional activation of stg/cdc25 and consequently cell cycle progression. Similarly, depletion of yeast TAF5, human TAF2, or a 2-fold reduction in chicken TBP result in G2/M arrest (26-28). Like TAF1, TBP regulates stg/cdc25 expression, providing support for the finding that the H2B-S33 kinase activity of TAF1 occurs in the context of TFIID (28). Interestingly, depletion of yeast TAF1, which does not possess a CTK, and inactivation of TAF1 HAT activity induce G1 arrest due to reduced transcription of B- and D-type cyclins, respectively (26, 29). Thus, loss of all TAF1 activities causes G2/M arrest while loss of TAF1 HAT activity causes G1 arrest, suggesting gene-specific requirements for TAF1 CTK and HAT activities. In contrast, the presence of phosphorylated H2B-S33 and acetylated H3 and H4 at the stg and gt promoters implies that TAF1 CTK and HAT activities can cooperate in transcriptional activation of some genes. This proposal is supported by the finding that loss of H2B-S33P from the gt promoter results in reduced transcription, despite the presence of TAF1-mediated histone acetylation. Thus, TAF1-mediated phosphorylation of H2B-S33 appears to work in concert with other TAF1-mediated histone modifications, H1 ubiquitination and H3 and H4 acetylation, to contribute to the chromatin-based mechanisms underlying transcription activation of eukaryotic genes.

Materials and Methods

Expression Plasmids

Baculovirus expression vectors expressing Flag(M2)-epitope tagged TAF1-derivatives were constructed by inserting TAF1 cDNA fragments into pVLFlag (31). DNA encoding the CTK and truncated derivatives were generated by PCR using primer pairs that insert a start codon at amino acid position 1496 (CTK) and 1593 (CTKΔ1600). The cDNA expressing CTKΔATP was generated by combining two PCR-generated cDNA fragments, which encode amino acids 1496-1747 and 1780-2132, respectively. The cDNA expressing TAF1ΔCTK was generated by site directed-mutagenesis using a primer pair that introduces a stop codon behind amino acid 1728. The point mutant D1538A was generated using the ‘Quick Change’ mutagenesis strategy (Stratagene). The following mutagenesis PCR primer and the corresponding complement were used, 5′-GAGATGTTCCTCGAGGCTCTCAAGCAGATTGTGGT-3′, with the mutated codon indicated in bold. Recombinant baculovirus containing the expression plasmids were generated using the Sapphire Baculovirus DNA positive selection vector (Orbigen) (15). The expression plasmid for RAP74 was generated as described (32).

Expression and Purification of Proteins

Flag(M2)-tagged TAF1 derivatives and RAP74 were expressed in Sf9 cells that had been infected with recombinant baculovirus as described (12). Recombinant proteins were immunoaffinity purified as described using Flag(M2)-epitope antibodies coupled to agarose (Sigma) (15). Nucleosomes and histone octamers were purified from 0-6 h Drosophila embryos as described (15, 16). Recombinant Xenopus laevis and Drosophila histones were expressed in E. coli and purified as described (3). Histone peptides used in this study were synthesized at the ZMBH (Heidelberg, Germany) or by PSL (Heidelberg, Germany). TFID was purified as described (32).

Acetylated-Lysine Interaction Assays

The interaction of CTK and CTK(D1548A) with unmodified and acetylated H4 peptides was monitored as described (14, 15). The acetylated H4 peptide carried acetyl-groups at lysine 5, 8, 12, and 16.

In Vitro Kinase Assays

Kinase reactions were programmed with 1 μg RAP74, 1 μg recombinant histone, 2 μg polynucleosomes, 2 μg histone octamers, or 300 ng histone peptide, and contained 400 ng endogenous TFIID or 250 ng recombinant TAF1 derivatives and 0.25 μCi γ³²P-ATP. Reactions were performed in 20 μl kinase buffer (25 mM Hepes (pH 7.6), 10% glycerol, and 400 mM NaCl) for 25 min at 25° C. Reaction products were analyzed by SDS-PAGE using 8%—(for recombinant proteins), 15%—(for histones), or 18% (for peptides) gels. After electrophoresis, gels were dried and exposed to X-ray film.

To identify new histone kinases, recombinant or highly purified general transcription factors (GTFs) or coactivators were incubated with the linker histone H1, the full complement of core histones, and γ³²P-ATP. Reaction products were separated by SDS-PAGE and radiolabeled histones were detected by autoradiography.

To quantify kinase reactions, reactions products were separated by SDS-PAGE and detected by coomassie blue staining. Radiolabeled H2B was detected by autoradiography, excised from the gel, and analyzed by scintillation counting. Compared to TFIID, TAF1 or CTK, the mutant CTK derivatives and TAF1ΔCTK exhibited reduced kinase activity (less than 0.1% of the activity determined for TFIID, TAF1 and CTK).

The PVDF-membrane kinase assay, to examine the activity of denatured/renatured TAF1, was performed as described by Pham and Sauer (2000) but with the following modifications (12). Recombinant TAF1 was separated by SDS-PAGE, electrophoretically transferred onto PVDF membrane (Schleicher and Schuell) and the position of TAF1 on the membrane was determined by Ponceau-S staining. TAF1 was subsequently denatured in guanidinium-HCl, renatured, and excised from the PVDF-membrane. The membrane was incubated with histones and γ³²P-ATP and the reaction products were detected as described above.

To measure the kinase activity of TFIID, TAF1, CTK, and mutant CTK-derivatives, reaction products of in vitro kinase assays were separated by SDS-PAGE and stained with coomassie blue. Radiolabeled H2B, H2BT and controls were excised from the gel and incorporation of radioactivity was determined by scintillation counting. Turnover values for phosphorylation of H2B-S33 were determined by measuring incorporation of radiolabeled phosphate groups into H2B in the presence of 3 μg TAF1, TFIID, or CTK-derivatives and 3 μg H2B or 12 μg nucleosomes. The turnover values were: TFIID (nucleosomes: 3.3/sec; H2B: 2.6/sec); TAF1: (nucleosomes: 2.5/sec; H2B: 2.5/sec); CTK: (nucleosomes: 2.6/sec; H2B: 4.4/sec).

RNAi and Flow Cytometry

RNAi and flow cytometry were carried out as described in Pile et al. (18). Plasmids used to generate TAF1 dsRNA were generated by PCR amplifying basepairs 3873-4879 from a TAF1 cDNA and cloning the product in both orientations into the pCRII-TOPO cloning vectors using the TOPO TA cloning kit (Invitrogen).

In Situ Hybridization on Whole Mount Drosophila Embryos

Flies were maintained using standard conditions (12). TAF1^{CTK} and cad/+; TAF1^{CTK} embryos were identified by the lack of GFP expression that is mediated by balancer chromosomes present in parental flies. In situ hybridization was performed as described (12), using in vitro transcribed, digoxigenin-labeled antisense gt RNA as probe.

H2B-S33P, TAF1, and H2B Antibodies

The H2B-S33P antiserum was generated in rabbits by repeated injection of an antigen comprised of acetylated BSA (Supercarrier, Perkin-Elmer) and an H2B peptide (amino acids 30-38), phosphorylated at S33 [KRKESpYAIY]. The serum was purified by a two-step immunoaffinity chromatography strategy. To eliminate antibodies that bind H2B but not H2B-S33P, the antiserum was incubated three times with a Sulfo-Link matrix (Pierce) containing H2BT that was prepared as described (15). Flow-through fractions were pooled and loaded on a Sulfo-Link matrix loaded with H2BT-S33P. Bound antibodies were eluted and concentrated using Protein-A agarose affinity-chromatography. Purified H2B-S33P antibody was used at a final dilution of 1:1000 for Western blot analysis. TAF1-M and TAF1-C rabbit polyclonal antibodies that recognize Drosophila TAF1 were generated against recombinant proteins encompassing amino acids 612-1140 and 1830-2132, respectively. The IgG fraction of the TAF1 serum was prepared using the Econo-Pac serum IgG purification kit (Bio-Rad). The SIN3 antibody is described in Pile and Wassarman (2000) (33). The H2B antibody recognizes an epitope in the COOH-terminus of H2B (Upstate). Immunoreactive signals were detected using the ECL+Plus Western blotting detection system (Amersham Biosciences) according the manufacturers instructions.

Mass-Spectrometry

To detect phosphorylation of H2B-S33, Drosophila histones were separated by SDS-PAGE and stained with colloidal coomassie. The protein band corresponding to H2B was excised from the gel and treated with trypsin. The resulting peptides were analyzed by MALDI-TOF mass-spectrometry. This analysis identified the phosphorylated H2B-peptide ESYAIYIYK (amino acids 32-40), indicating that H2B-S33 is phosphorylated in vivo.

In Vivo Cross-Linked Chromatin Immunoprecipitation (XChIP) and RT-PCR

RT-PCR was performed as described (15). Briefly, RNA was isolated from wild type and RNAi treated cells, reverse transcribed, and used as a template for PCR. PCR primer pairs that amplify nucleotides 95-636 of the string transcript and nucleotides 456-1014 of the actin5C transcript were used to examine transcription levels. XChIP was performed essentially as described (15). In vivo cross-linked chromatin was isolated from 1×10⁷ mock or RNAi treated cells, which had been incubated with 1.8% formaldehyde for 15 min. In vivo cross-linked chromatin was sheared to an average fragment length of 700 basepairs. To monitor histone modifications or the presence of proteins at the string promoter, chromatin isolated from 1×10⁶ cells was immunoprecipitated with the following antibodies: H2B-S33P (1:100 dilution), TAF1-M, (1:100 dilution), TAF1-C (1:1000), trimethyl-H3K9 (4 μg; Upstate), diacetyl-H3-K14 (4 μg; Upstate), acetyl-H4 (4 μg; Upstate), H3-S10P (4 μg; Upstate) and rabbit preimmune antiserum (5 μg). Chromatin:antibody complexes were purified by Protein-A agarose affinity-chromatography, incubated with RNase and Proteinase K to remove RNA and proteins, respectively, and incubated at 65° C. for 6 h to reverse the cross-links. Precipitated DNA was purified and used as a template for PCR. PCR primer pairs were used to amplify the string promoter region [(−157)-(+333)], the string coding region [(+165)-(+665)], the giant promoter region [(−387)-(+165)], the giant coding region [(+165)-(+565)], or the actin5C promoter [(−347)-(+155)]. PCR products were analyzed by gel electrophoresis using ethidium bromide containing agarose gels and detected by UV illumination.

Alternatively, Realtime PCR was used to detect the presence of the string or giant promoter in immunoprecipitated DNA pools. Realtime PCR was performed using an ABI 7700 instrument (Applied Biosystems). PCR was performed in the presence of SYBR green and analyzed using the ABI PRISM 7700 sequence detection system. The comparative C_{T} method was used to compare the presence of promoter fragments in TAF1, SIN3, and mock RNAi cells. In this case, the threshold cycle (C_{T}) represents the fractional cycle number at which two or more different PCR reactions amplify DNA at the exact same ratio.

### Example 2

### Phophorylation of Serine 33 in Drosophila melanogaster Histone H2B During the Cell Cycle

XChIP experiments were carried out as described above to detect phosphorylated H2B-S33 and TAF1 at the string promoter in G2/M-, G1-, and S-phase of Drosophila S2 cells. The results are shown in FIG. 12. Chromatin was prepared from 5×10⁶ sorted cells and immunoprecipitated using the indicated antibodies or rabbit pre-immune serum (control). PCR was used to detect a 500 bp genomic DNA fragment containing the string promoter in precipitated DNA pools. The results indicated that TAF1 and phosphorylated H2B-S33 are present at the string promoter in G2/M-phase.

### Example 3

### TAF1 Activates Transcription by Phophorylation of Serine 33 in Bovine and Human Histone H2B

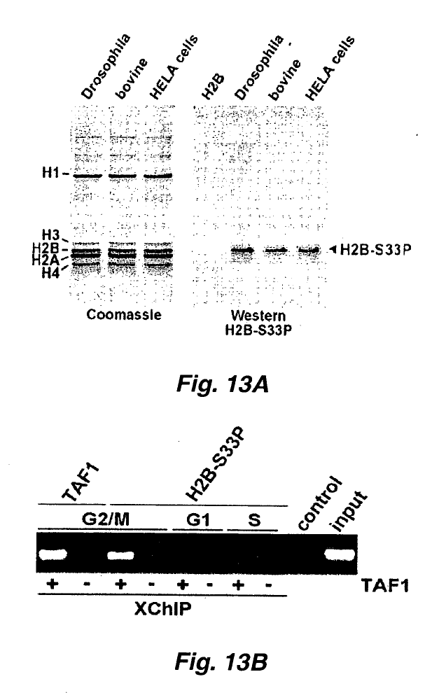

A Western Blot analysis was performed to determined whether the anti-H2B-S33P antibody described above detects the corresponding phosphorylated serine residue in H2B in histone preparations from bovine and human HELA cells. Referring to FIG. 13A, the left side of this figure shows a Coomassie stained SDS-page polyacrylamide gel showing histones prepared from Drosophila, bovine calf, and HELA cells. The right side of FIG. 13A shows a Western Blot analysis of recombinant Drosophila H2B, and histone preparations from Drosophila, calf, and HELA cells. Proteins were separated by SDS-PAGE, transferred onto nitrocellulose, and detected using the antibody recognizing phosphorylated H2B-S33 (phorphorylated H2B-S36, in human H2B). The results indicated that the serine corresponding to serine 33 in Drosophila H2B is also phosphorylated in bovine and human cells and is recognized by the antibody produced against the Drosophila protein.

XChIP experiments were carried out as described above to detect phosphorylated H2B-S36 and TAF1 at the human cdc25 promoter in human HELA cells. The results are shown in FIG. 13B. In vivo cross-linked chromatin was isolated from wild type HELA cells (+) and HELA cells lacking TAF1 due to RNAi (−) in G2/M, G1 and S phase. The chromatin was immunoprecipitated using the indicated antibodies or rabbit preimmune serum (control). FIG. 13B shows a photograph of an ethidium bromide stained agarose gel showing PCR products for the human cdc25 promoter in sorted wild-type HELA cells (+) and HELA cells lacking TAF1 due to RNAi (−). The results showed that TAF1 and phosphorylated H2B-S36 are present at the human cdc25 promoter in G2/M-phase in wild-type HELA cells, but not in HELA cells that lacked TAF1.

### Example 4

### Monoclonal Antibodies Specific for Phophorylated Serine 33 in Drosophila melanogaster Histone H2B

Monoclonal antibodies were generated as described above with the following modifications. The antigen employed was a bioconjugate consisting of acetylated-BSA coupled to a H2B peptide (H2BA-S33P). H2BA-S33p consists of amino acids 27-37 of H2B and contains phosphorylated serine 33. The antigen was injected into rats. The spleen of immunized rats was isolated and fused with SHFP-1 cells Hybridomas were screened for production of antibody that bound to H2BT-S33P but not H2BT by using Western blot and ELISA analysis to determine the elementary reaction pattern of the mAb of interest.

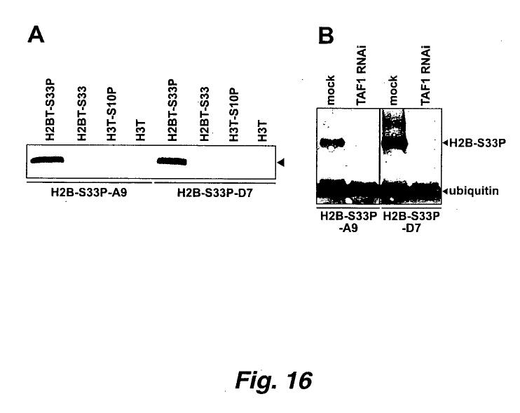

The Western blot results are shown in FIG. 16. Panel A shows the a Western blot of H2BT (amino acids 1-39), H2BT-S33P peptide that is phosphorylated at S33, H3T peptide (amino acids amino acids 1-32), and H3T-S10P peptide that is phosphorylated at S10 and S28. Phosphorylation of H2B-S33 was monitored by Western blot using the rat monoclonal antibodies anti-H2B-S33P-A9 and anti-H2B-S33P-D7. The position of the immuno-reactive signal corresponding to H2BT-S33P is indicated (arrowhead). Panel B shows a Western blot of histone octamers purified from Drosophila S2 cells incubated with mock double stranded RNA (mock) or double stranded RNA targeting the transcript of TAF1 (TAF1 RNAi). The Western blot was probed with anti-H2B-S33P-A9 (left) and anti-H2B-S33P-D7 (right) antibodies, and an antibody recognizing ubiquitin as a loading control. The positions of H2B phosphorylated at serine 33 (H2B-S33P) and ubiquitin are indicated.

### REFERENCES

1. B. Lemon, R. Tjian, Genes Dev. 20, 2551 (2000).
2. G. Orphanides, D. Reinberg, Cell 108, 439 (2002).
3. K. Luger, A. W. Mader, R. Richmond, D. F. Sargent, T. J. Richmond, Nature 389, 251 (1997).
4. M. G. Goll, T. H. Bestor, Genes Dev. 16, 1739 (2002).
5. W. Fischle, Y. Wang, C. D. Allis, Nature 425, 475 (2003).
6. W. L. Cheung, et al., Cell 113, 507 (2003).
7. I. Isenberg, Annu. Rev. Biochem. 48, 159 (1979).
8. Materials and Methods are detailed in the next section.
9. B. S. Chen, M. Hampsey, Curr. Biol. 12, R620 (2002).
10. R. Dikstein, S. Ruppert, R. Tjian, Cell 84, 781 (1996).
11. D. A. Wassarman, F. Sauer, J. Cell Sci. 114, 2895 (2001).
12. A.-D. Pham, F. Sauer, Science 289, 2357 (2000).
13. S. C. Elgin, J. Schilling, L. E. Hood, Biochemistry 18, 5679 (1979).
14. R. H. Jacobson, A. G. Ladurner, D. S. King, R. Tjian, Science 288, 1422 (2000).
15. C. Beisel, A. Imhof, J. Greene, E. Kremmer, F. Sauer, Nature 419, 857 (2002).
16. P. T. Georgel, T. Tsukiyama, C. Wu, EMBO J. 16, 4717 (1997).
17. M. S. Lee et al., Mol. Biol. Cell 3, 73 (1992).
18. L. A. Pile, E. M. Schlag, D. A. Wassarman, Mol. Cell. Biol. 22, 4965 (2002).
19. C. A. Mizzen, et al., Cell 87, 1261 (1996).
20. R. Rivera-Pomar, H. Jäckle, Trends Genet. 12, 478-483 (1996).
21. K. Luger, T. J. Richmond, Curr. Opin. Struct. Biol. 8, 33 (1998).
22. J. Recht, M. A. Osley, EMBO J. 18, 229 (1999).
23. A. Hamiche, J.-G. Kang, C. Dennis, H. Xiao, C. Wu., Proc. Natl. Acad. Sci. USA. 98, 14316 (2001).
24. L. Zheng, M. M. Zhou, FEBS Lett. 513, 124 (2002).
25. B. Florence, D. V. Faller, Front. Biosci. 6, D1008 (2001).
26. L. M. Apone, C. M. Verasius, J. C. Reese, M. R. Green, Genes Dev. 10, 2368 (1996).
27. J. Martin, R. Halenbeck, J. Kaufmann, Mol. Cell. Biol. 19, 5548 (1999).
28. M. Um, J. Yamauchi, S. Kato, J. L. Manley, Mol. Cell. Biol. 21, 2435 (2002).
29. E. L. Dunphy, T. Johnson, S. S. Auerbach, E. H. Wang, Mol. Cell. Biol. 20, 1134 (2000).
30. This work was supported by an NIH grant (GM066204-02) to D.A.W. and a Volkswagen Stiftung grant (I/77 996) to F.S.
31. S. Lichtsteiner, R. Tjian, EMBO J. 14, 3937(1995).
32. S. K. Hansen, R. Tjian, Cell 82, 565(1995).
33. L. A. Pile, D. A. Wassarman, EMBO J. 19, 6131 (2000).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of characterizing the proliferative state of cells in a biological sample, said method comprising detection of phosphorylation of histone H2B (H2B) at residue 36, wherein H2B residues are numbered according to human H2B, and wherein phosphorylation of H2B residue 36 is an indicator of cell proliferation.

2. The method of claim 1, wherein detection of phosphorylation of H2B at residue 36 comprises:
contacting a biological sample comprising H2B with an antibody specific for H2B comprising a phosphate on H2B residue 36 under conditions suitable for antibody binding; and
detecting H2B antibody binding.

3. The method of claim 2, additionally comprising determining whether H2B that is phosphorylated at H2B residue 36 (H2B-r36p) is physically associated with the promoter of a gene.

4. The method of claim 3, wherein the determination of whether H2B-r36p is physically associated with the promoter of a gene comprises in vivo cross-linked chromatin immunoprecipitation.

5. The method of claim 3, wherein H2B antibody binding is detected as an indication of the level of transcriptional activation of one or more genes.

6. The method of claim 5, wherein H2B antibody binding is detected as an indication of the level of transcriptional activation of one or more cell cycle genes.

7. The method of claim 6, wherein H2B antibody binding is detected as an indication of the level of transcriptional activation of one or more genes encoding proteins that contribute to progression through the G2/M phase of the cell cycle.

8. The method of claim 7, wherein H2B antibody binding is detected as an indication of the level of transcriptional activation of the human cdc25 gene.

9. The method of claim 5, wherein H2B antibody binding is detected as an indication of the level of transcriptional activation of one or more genes that participate in tissue differentiation.

10. The method of claim 5, wherein H2B antibody binding is detected as an indication of the level of transcriptional activation of one or more oncogenes.

11. The method of claim 3, wherein the amount of H2B-r36p physically associated with the promoter in a test sample is compared with the amount of H2B-r36p physically associated with the promoter in a control sample.

12. The method of claim 11, wherein the test sample comprises a tissue biopsy.

13. The method of claim 12, wherein the test sample comprises tissue suspected of being cancerous.

14. The method of claim 13, wherein the difference between the amount of H2B-r36p physically associated with the promoter in a test sample and the amount of H2B-r36p physically associated with the promoter in a control sample provides a metric useful in the diagnosis and/or prognosis of cancer.

15. The method of claim 3, wherein H2B residue 36 is a serine.

16. The method of claim 3, wherein H2B residue 36 is a threonine.

17. The method of claim 3, wherein the biological sample comprises histone H3 (H3) and/or histone H4 (H4), said method additionally comprising detection of acetylation of histone H3 at residue 14, wherein H3 residues are numbered according to human H3, and/or acetylation of histone H4, wherein acetylation of H3 residue 14 and acetylation of H4 are indicators of cell proliferation.

18. The method of claim 17, wherein detection of acetylation comprises:
contacting the biological sample with an antibody specific for H3 comprising an acetyl group on H3 residue 14 and/or with an antibody specific for acetylated H4 under conditions suitable for antibody binding; and
detecting H3 and/or H4 antibody binding.

19. The method of claim 18, additionally comprising determining whether H3 that is acetylated at H3 residue 14 (H3-r14a) and/or acetylated H4 is/are physically associated with the promoter of a gene.

20. The method of claim 19, wherein the determination of whether H3-r14a and/or acetylated H4 is/are physically associated with the promoter of a gene comprises in vivo cross-linked chromatin immunoprecipitation.

21. The method of claim 19, wherein H3 and/or H4 antibody binding is detected as an indication of the level of transcriptional activation of one or more genes.

22. The method of claim 21, wherein H3 and/or H4 antibody binding is detected as an indication of the level of transcriptional activation of one or more cell cycle genes.

23. The method of claim 22, wherein H3 and/or H4 antibody binding is detected as an indication of the level of transcriptional activation of one or more genes encoding proteins that contribute to progression through the G1 or G2/M phase of the cell cycle.

24. The method of claim 23, wherein H3 and/or H4 antibody binding is detected as an indication of transcriptional activation of the human cdc25 gene.

25. The method of claim 21, wherein H3 and/or H4 antibody binding is detected as an indication of the level of transcriptional activation of one or more genes that participate in tissue differentiation.

26. The method of claim 21, wherein H3 and/or H4 antibody binding is detected as an indication of the level of transcriptional activation of one or more oncogenes.

27. The method of claim 19, wherein the amount of H3-r14a and/or acetylated H4 physically associated with the promoter in a test sample is compared with the amount of H3-r14a and/or acetylated H4 physically associated with the promoter in a control sample.

28. The method of claim 27, wherein the test sample comprises a tissue biopsy.

29. The method of claim 28, wherein the test sample comprises tissue suspected of being cancerous.

30. The method of claim 29, wherein the difference between the amount of H3-r14a and/or acetylated H4 physically associated with the promoter in a test sample and the amount of H3-r14a and/or acetylated H4 physically associated with the promoter in a control sample provides a metric useful in the diagnosis and/or prognosis of cancer.

31. The method of claim 19, wherein the biological sample is from a mammal.

32. The method of claim 31, wherein the biological sample is from a human.

33. A method of characterizing the transcriptional activity of a gene in a biological sample, said method comprising determining whether histone H2B (H2B) that is phosphorylated at H2B residue 36 (H2B-r36p) is physically associated with the promoter of the gene, wherein H2B residues are numbered according to human H2B, and wherein physical association of H2B-r36p with the promoter is an indicator of transcriptional activation of the gene.

34. The method of claim 33, wherein the determination of whether H2B-r36p is physically associated with the promoter of the gene comprises in vivo cross-linked chromatin immunoprecipitation.

35. The method of claim 33, wherein the amount of H2B-r36p physically associated with the promoter in a test sample is compared with the amount of H2B-r36p physically associated with the promoter in a control sample.

36. The method of claim 33, wherein the difference between the amount of H2B-r36p physically associated with the promoter in a test sample and the amount of H2B-r36p physically associated with the promoter in a control sample provides a metric useful in the diagnosis and/or prognosis of cancer.

37. The method of claim 33, wherein the biological sample comprises histone H3 (H3) and/or histone H4 (H4), said method additionally comprising determining whether H3 that is acetylated at H3 residue 14 (H3-r14a) and/or acetylated H4 is/are physically associated with the promoter of a gene, wherein H3 residues are numbered according to human H3, and wherein physical association of H3-r14a and/or acetylated H4 with the promoter is/are indicators of transcriptional activation.

38. The method of claim 37, wherein the determination of whether H3-r14a and/or acetylated H4 is/are physically associated with the promoter of a gene comprises in vivo cross-linked chromatin immunoprecipitation.

39. The method of claim 37, wherein the amount of H3-r14a and/or acetylated H4 physically associated with the promoter in a test sample is compared with the amount of H3-r14a and/or acetylated H4 physically associated with the promoter in a control sample.

40. The method of claim 37, wherein the difference between the amount of H3-r14a and/or acetylated H4 physically associated with the promoter in a test sample and the amount of H3-r14a and/or acetylated H4 physically associated with the promoter in a control sample provides a metric useful in the diagnosis and/or prognosis of cancer.

41. The method of claims 33 or 37, wherein the gene comprises a cell cycle gene.

42. The method of claims 33 or 37, wherein the gene encodes a protein that contributes to progression through the G1 or G2/M phase of the cell cycle.

43. The method of claim 42, wherein the gene comprises the human cdc25 gene.

44. The method of claims 33 or 37, wherein the gene comprises a gene that participate in tissue differentiation.

45. The method of claims 33 or 37, wherein the gene comprises an oncogene.

46. The method of claims 33 or 37, wherein the biological sample is from a mammal.

47. The method of claim 46, wherein the biological sample is from a human.

48. A method for identifying one or more eukaryotic promoters in a biological sample, said method comprising isolating from the sample one or more polynucleotides that are physically associated in vivo with histone H2B (H2B) that is phosphorylated at H2B residue 36 (H2B-r36p), wherein H2B residues are numbered according to human H2B.

49. The method of claim 48, wherein the isolation of one or more polynucleotides that are physically associated in vivo with H2B-r36p comprises in vivo cross-linked chromatin immunoprecipitation.

50. The method of claim 48, wherein a plurality of different polynucleotides are isolated and are further characterized by hybridization to one or more known polynucleotides.

51. The method of claim 50, wherein the one or more known polynucleotides are arrayed in a DNA microarray.

52. The method of claim 48, wherein the biological sample is from a mammal.

53. The method of claim 52, wherein the biological sample is from a human.

54. An antibody specific for histone H2B (H2B) comprising a phosphate on H2B residue 36, wherein H2B residues are numbered according to human H2B.

55. The antibody of claim 54, wherein H2B residue 36 is a serine.

56. The antibody of claim 54, wherein H2B residue 36 is a threonine.

57. The antibody of claim 54, wherein the antibody comprises a polyclonal antibody.

58. The antibody of claim 54, wherein the antibody comprises a monoclonal antibody.

59. The antibody of claim 54, wherein the antibody comprises an IgG.

60. The antibody of claim 54, wherein the antibody comprises a Fab.

61. The antibody of claim 54, wherein the antibody comprises a (Fab′)₂.

62. The antibody of claim 54, wherein the antibody comprises a single, chain Fv (scFv).

63. The antibody of claim 54, wherein the antibody comprises a (scFv′)₂.

64. The antibody of claim 54, wherein the antibody binds to a mammalian H2B.

65. The antibody of claim 64, wherein the antibody binds to human H2B.

66. A method of producing the antibody of claim 54, the method comprising:
(a) administering a histone H2B (H2B), or a fragment thereof, to a mammal, wherein the H2B or H2B fragment comprises phosphorylated H2B residue 36, the H2B residues being numbered according to human H2B, and wherein said administration elicits an immune response; and
(b) recovering an antiserum or spleen cells from the mammal.

67. The method of claim 66, wherein spleen cells are recovered and used to produce one or more hybridomas.

68. A kit comprising a first article comprising the antibody of claim 54.

69. The kit of claim 68, additionally comprising instructions for carrying out the method of claim 2.

70. The kit of claim 68, additionally comprising a second article comprising a histone H2B (H2B), or a fragment thereof, wherein the H2B or H2B fragment comprises phosphorylated H2B residue 36, the H2B residues being numbered according to human H2B.

71. The kit of claim 68, additionally comprising a second article comprising:
an antibody specific for histone H3 (H3) comprising an acetyl group on H3 residue 14, wherein H3 residues are numbered according to human H3; and/or
an antibody specific for acetylated histone H4.

72. The kit of claim 71, additionally comprising a third article comprising:
a histone H3 (H3), or a fragment thereof, wherein the H3 or H3 fragment comprises acetylated H3 residue 14, the H3 residues being numbered according to human H3; and/or
an acetylated histone H4 or a fragment thereof.

73. An isolated polypeptide comprising a fragment of a transcription factor, wherein the fragment comprises a double bromodomain kinase and phosphorylates histone H2B (H2B) at H2B residue 36, wherein H2B residues are numbered according to human H2B, and wherein the polypeptide does not comprise more than about 650 contiguous amino acids of the transcription factor.

74. The polypeptide of claim 73, wherein H2B residue 36 is a serine.

75. The polypeptide of claim 73, wherein H2B residue 36 is a threonine.

76. The polypeptide of claim 73, wherein the transcription factor fragment comprises a serine/threonine kinase domain and a double ATP binding motif.

77. The polypeptide of claim 73, wherein the transcription factor is a mammalian transcription factor.

78. The polypeptide of claim 77, wherein the transcription factor is human TAF1.

79. The polypeptide of claim 78, wherein the transcription factor fragment comprises amino acids 1427-1893 of human TAF1.

80. An isolated polypeptide comprising a fragment of a histone H2B (H2B), wherein the fragment comprises H2B residue 36, the H2B residues being numbered according to human H2B, and wherein the polypeptide does not comprise more than about 40 contiguous amino acids of the H2B.

81. The polypeptide of claim 80, wherein H2B residue 36 is a serine.

82. The polypeptide of claim 80, wherein H2B residue 36 is a threonine.

83. The polypeptide of claim 80, wherein the H2B is a mammalian H2B.

84. The polypeptide of claim 83, wherein the H2B is human H2B.

85. The polypeptide of claim 80, wherein the polypeptide does not comprise more than about 10 contiguous amino acids of the H2B.

86. The polypeptide of claim 85, wherein the H2B fragment comprises the amino acid sequence KRKESYAIY (SEQ ID NO:______).

87. The polypeptide of claim 85, wherein the H2B fragment comprises the amino acid sequence SRKESYSIY (SEQ ID NO:______).

88. The polypeptide of claim 80, wherein H2B residue 36 is phosphorylated.

89. An isolated polynucleotide that encodes the polypeptide of claim 73, wherein the polynucleotide does not comprise more than about 1950 contiguous nucleotides of transcription factor coding sequence.

90. An isolated polynucleotide that encodes the polypeptide of claim 80, wherein the polynucleotide does not comprise more than about 120 contiguous nucleotides of histone H2B coding sequence.

91. A vector that comprises that polynucleotide of claims 89 or 90.

92. The vector of claim 91, wherein the vector is an expression vector.

93. A host cell comprising the vector of claim 91.

94. A host cell comprising the vector of claim 92.

95. A method of producing a polypeptide comprising:
(a) culturing the host cell of claim 94 under conditions suitable for expression of the polypeptide; and
(b) recovering the expressed polypeptide from the culture.

96. A method of prescreening for a modulator of phosphorylation of histone H2B (H2B) at residue 36 or a modulator of promoter association of H2B that is phosphorylated at residue 36 (H2B-r36p), said method comprising:
a) contacting a test agent with a polypeptide selected from the group consisting of an H2B, or a fragment thereof, or a transcription factor, or a fragment thereof, or with a polynucleotide encoding the polypeptide, wherein
(i) the H2B fragment comprises H2B residue 36, the H2B residues being numbered according to human H2B,
(ii) the transcription factor fragment comprises a double bromodomain kinase and phosphorylates H2B at H2B residue 36; and
b) detecting specific binding of the test agent to the polypeptide or polynucleotide.

97. The prescreening method of claim 96, wherein said method additionally comprises recording any test agent that specifically binds to the polypeptide or polynucleotide in a database of candidate agents that may modulate phosphorylation of H2B at residue 36 or promoter association of H2B-r36p.

98. The prescreening method of claim 96, wherein said detecting comprises detecting specific binding of the test agent to the polypeptide.

99. The prescreening method of claim 96, wherein said detecting comprises detecting specific binding of the test agent to the polynucleotide.

100. The prescreening method of claim 96, wherein said contacting is in vitro.

101. A method of screening for a modulator of phosphorylation of histone H2B (H2B) at residue 36, said method comprising:
(a) contacting a test agent with a medium comprising H2B, or a fragment thereof, and a transcription factor, or a fragment thereof, wherein
(i) the H2B fragment comprises H2B residue 36, the H2B residues being numbered according to human H2B,
(ii) the transcription factor fragment comprises a double bromodomain kinase and phosphorylates H2B at H2B residue 36, and
(iii) said contacting is carried out under conditions suitable for phosphorylation of H2B residue 36; and
(b) detecting phosphorylation of H2B residue 36.

102. The method of claim 101, wherein any phosphorylation of H2B residue 36 is compared with phosphorylation of H2B residue 36 in the absence of test agent or in the presence of a lower amount of test agent than in (a).

103. The method of claim 101, wherein the detection of phosphorylation of H2B at residue 36 comprises:
(i) contacting the H2B, or fragment thereof, with an antibody specific for H2B comprising a phosphate on H2B residue 36 under conditions suitable for antibody binding; and
(ii) detecting antibody binding.

104. A method of screening for a modulator of promoter association of histone H2B (H2B) that is phosphorylated at residue 36 (H2B-r36p), said method comprising:
(a) contacting a test agent with cells comprising H2B, or a fragment thereof, and a transcription factor, or a fragment thereof, wherein
(i) the H2B fragment comprises H2B residue 36, the H2B residues being numbered according to human H2B,
(ii) the transcription factor fragment comprises a double bromodomain kinase and phosphorylates H2B at H2B residue 36, and
(b) determining whether H2B-r36p is physically associated with the promoter of a gene.

105. The method of claim 104, wherein any promoter association of H2B-r36p is compared with the promoter association of H2B-r36p in the absence of test agent or in the presence of a lower amount of test agent than in (a).

106. The method of claim 104, wherein the determination of whether H2B-r36p is physically associated with the promoter of a gene comprises in vivo cross-linked chromatin immunoprecipitation.

107. The method of claim 106, wherein the gene is selected from the group consisting of: a cell cycle gene, a tissue differentiation gene, and an oncogene.

108. The method of claims 96, 101, or 104, wherein H2B residue 36 is a serine.

109. The method of claim 96, 101, or 104, wherein H2B residue 36 is a threonine.

110. The method of claim 96, 101, or 104, wherein the H2B is from a mammal.

111. The method of claim 110, wherein the H2B is from a human.

112. The method of claims 96, 101, or 104, wherein the transcription factor is from a mammal.

113. The method of claim 112, wherein the transcription factor is from a human.

114. The method of claim 101, wherein said method additionally comprises recording any inhibitor of phosphorylation of H2B at residue 36 or any inhibitor of promoter association of H2B-r36p in a database of candidate agents that may inhibit cell proliferation.

115. The method of claim 101, wherein said method additionally comprises determining whether the test agent inhibits cell proliferation.

116. The method of claim 101, wherein said method additionally comprises recording any test agent that stimulates phosphorylation of H2B at residue 36 or that stimulates promoter association of H2B-r36p in a database of candidate agents that may stimulate cell proliferation.

117. The method of claim 101, wherein said method additionally comprises determining whether the test agent stimulates cell proliferation.

118. A method of modulating cell proliferation, the method comprising contacting cells comprising a transcription factor and histone H2B (H2B) with an effective amount of a modulator, wherein:
the transcription factor comprises a double bromodomain kinase and phosphorylates H2B at H2B residue 36, wherein H2B residues are numbered according to human H2B
the modulator reduces or increase said phosphorylation; and
an effective amount is an amount sufficient to inhibit or stimulate, respectively, cell proliferation.

119. The method of claim 118, wherein the cells are mammalian cells.

120. The method of claim 119, wherein the cells are human cells.

121. The method of claim 120, wherein the transcription factor is TAF1.

122. The method of claim 118, wherein the cells are in vitro.

123. The method of claim 118, wherein the cells are in vivo.

124. The method of claim 118, wherein the modulator reduces said phosphorylation.

125. The method of claim 124, wherein said contacting is performed by administering a composition comprising the modulator to a cancer patient.

126. The method of claim 118, wherein the modulator increases said phosphorylation.

127. The method of claim 126, wherein said contacting is performed by administering a composition comprising the modulator to a subject having a condition treatable by induction of cell proliferation.
